(19) United States
(12) Patent Application Publication
(10) Pub. No.: US 2016/0160299 A1
(43) Pub. Date: Jun. 9, 2016

(54) SHORT EXOGENOUS PROMOTER FOR HIGH LEVEL EXPRESSION IN FUNGI

(71) Applicant: Board of Regents, The University of Texas System, Austin, TX (US)

(72) Inventors: Hal Alper, Austin, TX (US); Heidi Redden, Austin, TX (US)

(21) Appl. No.: 14/930,322

(22) Filed: Nov. 2, 2015

Related U.S. Application Data
(60) Provisional application No. 62/073,318, filed on Oct. 31, 2014.

(51) Int. Cl. C12Q 1/68; C12N 15/80

(52) U.S. Cl. CPC C12Q 1/6897; C12N 15/80

(57) ABSTRACT

Provided herein are short exogenous fungi transcription promoter nucleic acid sequences and methods of using the exogenous fungi transcription promoter nucleic acid sequences to modulate transcription initiation or rate of transcription.

### CROSS-REFERENCES TO RELATED APPLICATIONS

This application claims the benefit of U.S. Provisional Application No. 62/073,318, filed Oct. 31, 2014, the content of which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant number R01 GM090221-03, awarded by the National Institutes of Health and grant number FA9550-14-1-0089 awarded by the Air Force Office of Scientific Research. The government has certain rights in this invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 48932-526001US ST25.TXT, created November 2, 10,515 bytes, machine format IBM-PC, MS-Windows operating system, is hereby incorporated by reference.

### BACKGROUND OF THE INVENTION

Tunable control of flux through a given pathway is useful in metabolic engineering. Promoters play a crucial part in synthetic biology, by not just allowing overexpression of a gene, but also, providing the ability to tune enzymatic activity (by altering enzyme abundance) of every step in a pathway. However, successful design strategies for yeast promoters are limited. For decades, error-prone PCR mutagenesis on native promoters has been used to create synthetic promoters. But such promoters result in high homology to the native template. These methods all result in promoters of either the same length as the original, or in some cases, longer. Thus, there is a need in the art for short promoters in fungi for at least metabolic engineering procedures. Provided herein are solutions to these and other problems in the art.

### BRIEF SUMMARY OF THE INVENTION

Provided herein, inter alia, are short exogenous promoter nucleic acid sequences and methods of using the exogenous promoter nucleic acid sequences to modulate transcription initiation or rate of transcription. These short promoters may initiate transcription or modulate the rate of transcription with both significantly shorter sequences (thus saving on the amount of DNA used in an expression cassette) and with diverse sequences (thus preventing homologous recombination with native promoters).

In one aspect is an exogenous fungi transcription promoter nucleic acid sequence that includes an upstream activating nucleic acid sequence, a core promoter nucleic acid sequence, and an upstream spacer nucleic acid sequence linking the upstream activating nucleic acid sequence to the core promoter nucleic acid sequence. The core promoter nucleic acid sequence includes a fungi TATA box sequence motif, a fungi transcription start site nucleic acid sequence, and a core promoter linker sequence linking the fungi TATA box sequence motif and the fungi transcription start site nucleic acid sequence.

Also provided herein are fungi cells which include an exogenous fungi transcription promoter nucleic acid sequence described herein.

Further provided herein are expression constructs which include an exogenous fungi transcription promoter nucleic acid sequence described herein.

Provided herein are methods expressing a gene in a fungi cell. In one aspect is a method of expressing a gene in a fungi cell by transforming the fungi cell with an expression construct described herein that includes a gene operably connected to an exogenous fungi transcription promoter nucleic acid sequence described herein and allowing the cell to express the expression construct, where the exogenous fungi transcription promoter nucleic acid sequence modulates a level of transcription initiation or a rate of transcription of the gene, thereby expressing the gene in the fungi cell. In another aspect is a method of modulating expression of an endogenous gene in a fungi cell by operably linking an exogenous fungi transcription promoter nucleic acid sequence into a genome of the fungi cell, where the exogenous fungi transcription promoter nucleic acid sequence modulates a level of transcription initiation or a rate of transcription of the gene, thereby expressing the gene in the fungi cell.

Also provided herein are methods of testing a fungi core promoter nucleic acid sequence. In one aspect is a method of testing a fungi core promoter nucleic acid test sequence by determining a level of transcription initiation or a rate of transcription of a core promoter nucleic acid test sequence. The core promoter nucleic acid test sequence includes a fungi TATA box sequence motif, a fungi transcription start site nucleic acid sequence, and a core promoter linker test sequence.

Further provided herein are methods of testing an upstream activating nucleic acid sequence. In one aspect is a method of testing an upstream activating nucleic acid test sequence by determining a level of transcription initiation or a rate of transcription of a fungi transcription promoter nucleic acid test sequence that includes a non-native upstream activating nucleic acid test sequence, a fungi promoter sequence, and an upstream spacer nucleic acid test sequence which links the non-native upstream activating nucleic acid test sequence and the fungi promoter sequence.

### BRIEF DESCRIPTION OF THE DRAWINGS

FIGS. 1A-1B. FIG. 1A depicts as a cartoon an overview of methods disclosed herein. Twenty-seven libraries including 15 million candidates were created. 0.15% of the most promising libraries were sorted by fluorescence activated cell sorting (FACS). These sorted cells were plated and colonies were picked to determine fluorescence strength. High expressing candidates were sequenced. 19 strong promoters were present in the pool of 82 sequenced candidates. These 19 strong promoters were characterized under CLB activation, gal binding site (i.e. a GAL4 upstream activating nucleic acid sequence) (GBS) activation and with just the core. FIG. 1B depicts as a cartoon that one library of 1.3 million UAS candidates were sorted and plated. Of these, 120 colonies' fluorescence was assessed by flow cytometry, resulting in 5 strong UAS candidates.

FIG. 2. A histogram of results of activation studies for UAS_{CIT} (SEQ ID NO:18) and UAS_{CLB} (SEQ ID NO:19).

FIGS. 3A-3B. FIG. 3A is a cartoon representations of promoter disclosed herein, and FIG. 3B is a histogram of results employing indicated promoters. Cores can be used to create inducible promoters. Cores were paired with a gal binding site (GBS). In the presence of galactose, promoters are induced. In some promoter pairings, promoter strength was that of full native galactose promoter, but at a fraction of the length as shown in the scaled illustrations. Y-axis: observed fluorescence (AU). For each histogram bin pair, entries are in the order glucose (left) and galactose (right). Legend (left to right): GBS 1; GBS 2; GBS 3 (SEQ ID NO:16); GBS 4 (SEQ ID NO:17); GBS 5; GBS 6; GBS 7; GBS 8; GBS 9; full native galactose and Leu min.

FIGS. 4A-4B: FIG. 4A depicts that cores are very distinct from one another, spanning a % GC content of 47 to 73. The quantity, quality and orientation of transcription factor binding sites (TFBS) as determined by YEASTRACT database varies greatly. TFBS are indicated by arrows with direction of arrow designating direction of site. Sequence legend (top to bottom, corresponding to core 1 to 9, respectively): SEQ ID NOS:20-28. FIG. 4B depicts N10 sequence and spacer sequences for UASA (SEQ ID NO:10), UASB (SEQ ID NO:11), UASC (SEQ ID NO:12), UASD (SEQ ID NO:13), UASE (SEQ ID NO:14), and UASF (SEQ ID NO:15). Sequence legend (top to bottom, corresponding to sequences including UASA to UASF, respectively): SEQ ID NOS: 29-34.

FIGS. 5A-5B. FIG. 5A depicts histogram showing that 10 nt UAS derived from core 1 library can be combined with core 2 to yield functioning promoters. 10 nt UAS can be placed in tandem to yield increasingly stronger promoters. Legend (left to right): no yECitrine, spacer-core3; UASA (SEQ ID NO:10) core1 (SEQ ID NO:1); UASB (SEQ ID NO:11) core1 (SEQ ID NO:1); UASC (SEQ ID NO:12) core1 (SEQ ID NO:1); UASA (SEQ ID NO:10), UASB (SEQ ID NO:11) core1 (SEQ ID NO:1); UASCIT (SEQ ID NO:18) core1 (SEQ ID NO:1); Cyc, spacer=core2 (SEQ ID NO:2); UASA (SEQ ID NO:10) core3 (SEQ ID NO:3); UASB (SEQ ID NO:11) core 3 (SEQ ID NO:3); UASB (SEQ ID NO:11) core2 (SEQ ID NO:2); and UASCIT (SEQ ID NO:18) core2 (SEQ ID NO:2). FIG. 5B depicts histogram of results of additional data for the combination of hybrid promoter elements for the synthetic promoters discloses herein. Legend (left to right): core3, spacercore3, 101core3, 109core3, 19core3, 109core3, 101-109-19core3, citcore3, clbcore3, core9, spacercore9, 101core9, 109core9, 19core9, 101-19core9, 109-19core9, 101-109-19core9, citcore9, clbcore9, cyc, no yECitrine, and GPD.

FIGS. 6A-6B. FIG. 6A depicts representative synthetic hybrid assembled UAS sequences that activate core elements to yield high strength constitutive promoters. The length of the promoters are illustrated to scale. All synthetic UAS sequences shown (UAS_{F}, UAS_{E} and UAS_{C}) are positioned upstream of core element using AT-rich neutral 30 bp spacer. FIG. 6B depicts histogram of fluorescence activity with indicated promoters, in order (left to right): no yECitrine, core 1, UAS_{F}-Core 1, UAS_{E}-Core 1, UAS_{C}-Core 1, UAS_{F-E-c}-Core 1, CYC1, and GPD (TDH3).

### DETAILED DESCRIPTION OF THE INVENTION

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and nucleic acid chemistry and hybridization described below are those well-known and commonly employed in the art. Standard techniques are known in the art and used for nucleic acid synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Nucleic acid as used herein also refers nucleic acids that have the same basic chemical structure as a naturally occurring nucleic acids. All sequences are written 5′- to 3′- unless otherwise indicated.

The terms “DNA” and “RNA” refer to deoxyribonucleic acid and ribonucleic acid, respectively. The symbols “A,” “C,” “T,” “U,” and “G” are used herein according to their standard definitions and refer to adenine, cytosine, thymidine, and guanine respectively. The symbol “Y” is used herein according to its common definition in the art and refers to C or T. The symbol “W” is used herein according to its common definition in the art and refers to A or T. The symbol “R” is used herein according to its common definition in the art and refers to A or G. The symbol “N” is used herein according to its common definition in the art and refers to A, T, C, or G.

“Synthetic mRNA” as used herein refers to any mRNA derived through non-natural means such as standard oligonucleotide synthesis techniques or cloning techniques (i.e. non-native mRNA or exogenous mRNA). Such mRNA may also include non-native derivatives of naturally occurring nucleotides. Additionally, “synthetic mRNA” herein also includes mRNA that has been expressed through recombinant techniques or exogenously, using any expression vehicle, including but not limited to prokaryotic cells, eukaryotic cell lines, and viral methods. “Synthetic mRNA” includes such mRNA that has been purified or otherwise obtained from an expression vehicle or system.

The words “complementary” or “complementarity” refer to the ability of a nucleic acid in a polynucleotide to form a base pair with another nucleic acid in a second polynucleotide. For example, the sequence A-G-T is complementary to the sequence T-C-A. For example, if a nucleobase at a certain position of nucleic acid is capable of hydrogen bonding with a nucleobase at a certain position of another nucleic acid, then the position of hydrogen bonding between the two nucleic acids is considered to be a complementary position. Nucleic acids are “substantially complementary” to each other when a sufficient number of complementary positions in each molecule are occupied by nucleobases that can hydrogen bond with each other. Thus, the term “substantially complementary” is used to indicate a sufficient degree of precise pairing over a sufficient number of nucleobases such that stable and specific binding occurs between the nucleic acids. The phrase “substantially complementary” thus means that there may be one or more mismatches between the nucleic acids when they are aligned, provided that stable and specific binding occurs. The term “mismatch” refers to a site at which a nucleobase in one nucleic acid and a nucleobase in another nucleic acid with which it is aligned are not complementary. The nucleic acids are “perfectly complementary” to each other when they are fully complementary across their entire length.

Where a method disclosed herein refers to “amplifying” a nucleic acid, the term “amplifying” refers to a process in which the nucleic acid is exposed to at least one round of extension, replication, or transcription in order to increase (e.g., exponentially increase) the number of copies (including complimentary copies) of the nucleic acid. The process can be iterative including multiple rounds of extension, replication, or transcription. Various nucleic acid amplification techniques are known in the art, such as PCR amplification or rolling circle amplification. Amplifying as used herein also refers to “gene synthesis” or “artificial gene synthesis” to create single-strand or double-strand polynucleotide sequences de novo using techniques known in the art.

A “primer” as used herein refers to a nucleic acid that is capable of hybridizing to a complimentary nucleic acid sequence in order to facilitate enzymatic extension, replication or transcription.

A “library” refers to a plurality of nucleic acid sequences (including those described herein) which are tested or screened for transcription initiation or transcription rate (i.e. promoter activity). A library may include nucleic acid sequences that share similar characteristics (e.g. length of a linker, composition of a linker, a TATA box sequence motif, or an upstream activating nucleic acid sequence). A library may include nucleic acid sequences that are randomly generated so long as the nucleic acid sequences include one or more of components of a core promoter nucleic acid sequence as described herein. Accordingly, a library may contain one or more regions of variation where the nucleotides and nucleotide positions can be Y, W, R, or N. Nucleic acid sequences of a library may be synthesized using methods known in the art or may be created using other techniques known in the art.

Nucleic acid is “operably linked” or “operably connected” when it is placed into a functional relationship with another nucleic acid sequence. For example, DNA encoding a promoter is operably linked to a coding sequence if it modulates the initiation of transcription of the sequence. Generally, “operably linked” means that the DNA sequences being linked are near each other, contiguous, and in reading phase. Operably linked therefore refers to a promoter that initiates transcription of a gene or modulates a rate of transcription of a gene.

The term “promoter” is used according to its plain ordinary meaning in the art and refers to a 5′ nucleic acid sequence at the start of an open reading frame required for initiation of transcription in a fungi cell. Promoters may recruit transcription binding factors or components of the pre-initiation complex necessary (PIC) to initiate transcription by RNA polymerase II (RNAP). A promoter may be a native promoter (e.g. a native yeast promoter) or an exogenous promoter (e.g. an exogenous fungi transcription promoter nucleic acid sequence described herein).

The term “transcription initiation” as used herein refers to the process of recruiting the PIC and beginning transcription of a gene product operably linked to a promoter. The term “transcription rate” as used herein refers to determining an amount of transcription of a gene product.

A “transcription factor binding site” is used according to its plain ordinary meaning in the art and refers to a nucleic acid sequence that binds to a transcription factor. Transcription factor binding sites may modulate the level of transcription initiation or the rate of transcription. Similarly, a “transcription factor” as used herein refers to a composition (e.g. protein, polynucleotides, or compound) which binds to a nucleic acid sequence (e.g. a promoter) to initiate or enhance transcription. A transcription factor binding site may be a consensus sequence or a non-consensus region that binds a particular transcription factor or set of transcription factors.

The term “exogenous fungi transcription promoter nucleic acid sequence” refers to a non-native fungi promoter sequence that modulates transcription initiation or rate of transcription when 5′ operably linked to a gene.

A “fungi TATA box sequence motif” is a nucleic acid sequence that binds and/or recruits transcription factors (e.g. the TATA binding protein) in a fungal cell. Typically, transcription factors begin the process of initiating transcription. A fungi TATA box sequence motif may be a nucleic acid sequence that is native to a fungi cell.

A “fungi transcription start site nucleic acid sequence” is used in accordance with its plain and ordinary meaning and refers to a nucleic acid sequence which signals or otherwise sets a location for transcription of a gene to occur in a fungal cell. The fungi transcription start site nucleic acid sequence may also demark the start of the 5′ untranslated region. Exemplary transcription start site nucleic acid sequences include those described in Zhang Z, Dietrich F, Nucleic Acids Res. 2005; 33(9): 2838-2851. A fungi transcription start site nucleic acid sequence may be a nucleic acid sequence that is native to a fungi cell.

The terms “core promoter,” “core promoter nucleic acid sequence,” “fungi core promoter,” and “fungi core promoter nucleic acid sequence” are used interchangeably herein and refer to a nucleotide sequence capable of binding the preinitiation complex (“PIC”) which typically includes transcription factors and a RNA polymerase (e.g. RNA polymerase II).

An “upstream activating nucleic acid sequence” or “UAS” is a nucleic acid sequence located 5′ to a promoter (e.g. a core promoter nucleic acid sequence described herein) which activates (e.g. increases activity of) the promoter (e.g. a core promoter nucleic acid sequence). A UAS may be the sole activator of a promoter (e.g. a core promoter nucleic acid sequence has little-to-no activity in the absence of the activator of the UAS) or may further activate or enhance the activity of a promoter. A UAS may be operably linked to a native promoter to modulate the expression of a native gene. A UAS may be inducible or constitutive as described herein. Exemplary upstream activating nucleic acid sequences include, but are not limited to, GAL4 upstream activating sequences (e.g. a UAS nucleic acid sequence capable of binding to GAL4 protein), CIT upstream activating sequences (e.g. a UAS nucleic acid sequence capable of binding to CIT), or CLB upstream activating sequences (e.g. a UAS nucleic acid sequence capable of binding to CLB). The term “UAS” in the context of a specific UAS may include optional appended indicia, wherein such indicia are optionally subscripted. Thus, the term “UASA,” “UAS_{A}” and the like are synonymous, referring to the UAS sequence of SEQ ID NO:10 disclosed herein.

The terms “GAL4 upstream activating sequence,” “GBS,” and “UAS_{GAL4}” are used interchangeably herein and refer to a truncated GAL4 upstream activating sequence, which shares homology to portion of a full-length GAL4 upstream activating sequence but is less than about 60%, 65%, 70%, 75%, 80%, 85%, 90%, or 95% of the length of the corresponding full-length full-length GAL4 upstream activating sequence. A GAL4 upstream activating sequence may be numbered (e.g. GBS1, GBS2, GBS3, GBS4 . . . ) where each numbered GAL4 upstream activating sequence represents a different truncated sequence. A GAL4 upstream activating sequence may have SEQ ID NO:16 or SEQ ID NO:17: CGGGCGACAGCCCTCCG (SEQ ID NO:16); CGGAAGACTCTCCTCCG (SEQ ID NO:17).

The terms “full-length GAL4 upstream activating sequence,” “full-native GAL4 upstream activating nucleic acid sequence,” and “full-length UAS_{GAL4}” refer to the native, full-length GAL4 upstream activating sequence.

The terms “CIT upstream activating sequence,” “UASCIT,” and “UAS_{CIT}” are used interchangeably herein and refer to a truncated CIT upstream activating sequence, which shares homology to portion of a full-length CIT upstream activating sequence but is less than about 60%, 65%, 70%, 75%, 80%, 85%, 90%, or 95% of the length of the corresponding full-length CIT upstream activating sequence. A CIT upstream activating sequence may have SEQ ID NO:18:

(SEQ ID NO: 18)
TAGAGATTACTACATATTCCAACAAGACCTTCGCAGGAAAGTATACCTAA
ACTAATTAAAGAAATCTCCGAAGTTCGCATTTCATTGAACGGCTCAATTA
ATCTTTGTAAATATGAGCGTTTTTACGTTCACATTGCCTTTTTTTTTATG
TATTTACCTTGCATTTTTGTGCTAAAAGGCGTCACGTTTTTTTCCGCCGC
AGCCGCCCGGAAATGAAAAGTATGACCCCCGCTAGACCAAAAATACTTTT
GTGTTATTGGAGGATCGCAATCCCT.

The terms “full-length CIT upstream activating sequence” and “full-length UAS_{CIT}” refer to the native, full-length CIT upstream activating sequence.

The terms “CLB upstream activating sequence” “UASCLB,” and “UAS_{CLB}” are used interchangeably herein and refer to a truncated CLB upstream activating sequence, which shares homology to portion of a full-length CLB upstream activating sequence but is less than about 60%, 65%, 70%, 75%, 80%, 85%, 90%, or 95% of the length of the corresponding full-length CLB upstream activating sequence. A CLB upstream activating sequence may have SEQ ID NO:19:

(SEQ ID NO: 19)
AGTGGAATTATTAGAATGACCACTACTCCTTCTAATCAAACACGCGGAAA
TAGCCGCCAAAAGACAGATTTTATTCCAAATGCGGGTAACTATTTGTATA
ATATGTTTACATATTGAGCCCGTTTAGGAAAGTGCAAGTTCAAGGCACTA
ATCAAAAAAGGAGATTTGTAAATATAGCGACCGAATCAGGAAAAGGTCAA
CAACGAAGTTCGCGATATGGATGAACTTCGGTGCCTGTCC.

The term “full-length CLB upstream activating sequence” and “full-length UAS_{CLB}” refer to the native, full-length CLB upstream activating sequence.

The phrase “test sequence” when used in connection with terms described herein (e.g. fungi core promoter or upstream activating nucleic acid), refers to an experimental nucleic acid sequence to test modulation of a promoter sequence activity (e.g. transcription initiation or rate of transcription). A test sequence may be a nucleic acid sequence having a different length or nucleotide composition than another test sequence or a control sequence (e.g. an exogenous fungi transcription promoter nucleic acid sequence or a native promoter).

The term “constitutive” is used accordingly to its plain ordinary meaning in the art and refers a nucleic acid sequence having promoter activity that is constant and active. The term “inducible” is used accordingly to its plain ordinary meaning in the art and refers to expression that occurs in response to an environmental stimulus or binding of a particular molecule (e.g. galactose, lactose, or a transcription factor).

“Heterologous” refers to a gene or its product (e.g. a mRNA) or polypeptide or protein translated from the gene product, which is not native to or otherwise typically not expressed by the host cell. Similarly “heterologously expressed” refers to expression of a non-native gene or gene product by a host cell (e.g. a fungi cell). A heterologous gene may be introduced into the host using techniques known in the art including, for example, transfection, transformation, or transduction.

The word “expression” or “expressed” as used herein in reference to a DNA nucleic acid sequence (e.g. a gene) means the transcriptional and/or translational product of that sequence. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). The level of expression of a DNA molecule may also be determined by the activity of the protein.

The terms “expression construct” and “expression vector,” are used interchangeably herein in accordance with their plain ordinary meaning and refer to a polynucleotide sequence engineered to introduce particular genes into a target cell. Expression constructs described herein can be manufactured synthetically or be partially or completely of biological origin, where a biological origin includes genetically based methods of manufacture of DNA sequences.

The term “gene” means the segment of DNA involved in producing a protein or non-coding RNA; it includes regions preceding and following the coding region (leader and trailer) as well as intervening sequences (introns) between individual coding segments (exons). The leader, the trailer as well as the introns include regulatory elements that are necessary during the transcription and the translation of a gene. A “protein gene product” is a protein expressed from a particular gene.

The term “modulator” refers to a composition (e.g. an exogenous fungi transcription promoter nucleic acid sequence) that increases or decreases the expression of a target molecule or which increases or decreases the level of or the efficiency of transcription initiation or rate of transcription in a gene. Modulator may also refer to a composition which increases or decreases the expression of a non-coding RNA. Modulator may refer to a molecule or composition required by an inducible promoter for activity.

The term “modulate” is used in accordance with its plain ordinary meaning and refers to the act of changing or varying one or more properties. For example, a promoter sequence modulates the expression of a target protein changes by increasing or decreasing a property (e.g. efficiency of) associated with transcription initiation or rate of transcription. An exogenous transcription promoter nucleic acid sequence described herein may modulate the expression of a non-coding RNA.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “isolated” refers to a nucleic acid, polynucleotide, polypeptide, protein, or other component that is partially or completely separated from components with which it is normally associated (other proteins, nucleic acids, cells, etc.).

A “yeast cell” as used herein, refers to a eukaryotic unicellular microorganism carrying out metabolic or other function sufficient to preserve or replicate its genomic DNA. Yeast cells referenced herein include, for example, the following species: Kluyveromyces lactis, Torulaspora delbrueckii, Zygosaccharomyces rouxii, Saccharomyces cerevisiae, Yarrowia lipolytica, Candida intermedia, Cryptococcos neoformans, Debaryomyces hansenii, Phaffia rhodozyma, or Scheffersomyces stipitis. A “recombinant yeast cell” is a yeast cell which includes and/or expresses an exogenous fungi transcription promoter nucleic acid sequence described herein.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. A control as used herein may refer to the absence of an exogenous fungi transcription promoter nucleic acid sequence described herein. A control may refer to expression of a gene using a native promoter.

### I. EXOGENOUS FUNGI TRANSCRIPTION PROMOTER NUCLEIC ACID SEQUENCES

Provided herein are exogenous fungi transcription promoter nucleic acid sequences. In one aspect is an exogenous fungi transcription promoter nucleic acid sequence that includes an upstream activating nucleic acid sequence, a core promoter nucleic acid sequence, and an upstream spacer nucleic acid sequence linking the upstream activating nucleic acid sequence to the core promoter nucleic acid sequence. The core promoter nucleic acid sequence includes a fungi TATA box sequence motif, a fungi transcription start site nucleic acid sequence, and a core promoter linker sequence linking the fungi TATA box sequence motif and the fungi transcription start site nucleic acid sequence.

The fungi TATA box sequence motif may have the sequence TATAW¹W²R, where W¹ and W² are independently adenine (A) or thymidine (T) and R is A or guanine (G). W¹ may be A. W¹ may be T. R may be A. R may be G. W¹ may be A where R is G. W¹ may be A where R is A. W² may be A where R is G. The fungi TATA box sequence motif may have the sequence TATAAAAG.

The core promoter nucleic acid linker sequence may be 10 to 50 nucleotides in length. The core promoter nucleic acid linker sequence may be 10 to 45 nucleotides in length. The core promoter nucleic acid linker sequence may be 10 to 40 nucleotides in length. The core promoter nucleic acid linker sequence may be 10 to 35 nucleotides in length. The core promoter nucleic acid linker sequence may be 10 to 30 nucleotides in length. The core promoter nucleic acid linker sequence may be 10 to 25 nucleotides in length. The core promoter nucleic acid linker sequence may be 10 to 20 nucleotides in length. The core promoter nucleic acid linker sequence may be 10 to 5 nucleotides in length. The core promoter nucleic acid linker sequence may be 15 to 50 nucleotides in length. The core promoter nucleic acid linker sequence may be 15 to 45 nucleotides in length. The core promoter nucleic acid linker sequence may be 15 to 40 nucleotides in length. The core promoter nucleic acid linker sequence may be 15 to 35 nucleotides in length. The core promoter nucleic acid linker sequence may be 15 to 30 nucleotides in length. The core promoter nucleic acid linker sequence may be 15 to 25 nucleotides in length.

The core promoter nucleic acid linker sequence may be 20 to 50 nucleotides in length. The core promoter nucleic acid linker sequence may be 20 to 45 nucleotides in length. The core promoter nucleic acid linker sequence may be 20 to 40 nucleotides in length. The core promoter nucleic acid linker sequence may be 20 to 35 nucleotides in length. The core promoter nucleic acid linker sequence may be 20 to 30 nucleotides in length. The core promoter nucleic acid linker sequence may be 20 to 25 nucleotides in length. The core promoter nucleic acid linker sequence may be 25 to 50 nucleotides in length. The core promoter nucleic acid linker sequence may be 25 to 45 nucleotides in length. The core promoter nucleic acid linker sequence may be 25 to 40 nucleotides in length. The core promoter nucleic acid linker sequence may be 25 to 35 nucleotides in length. The core promoter nucleic acid linker sequence may be 25 to 30 nucleotides in length. The core promoter nucleic acid linker sequence may be 30 to 50 nucleotides in length. The core promoter nucleic acid linker sequence may be 30 to 45 nucleotides in length. The core promoter nucleic acid linker sequence may be 30 to 40 nucleotides in length. The core promoter nucleic acid linker sequence may be 30 to 35 nucleotides in length.

The core promoter nucleic acid linker sequence may be 50 nucleotides in length. The core promoter nucleic acid linker sequence may be 45 nucleotides in length. The core promoter nucleic acid linker sequence may be 40 nucleotides in length. The core promoter nucleic acid linker sequence may be 39 nucleotides in length. The core promoter nucleic acid linker sequence may be 38 nucleotides in length. The core promoter nucleic acid linker sequence may be 37 nucleotides in length. The core promoter nucleic acid linker sequence may be 36 nucleotides in length. The core promoter nucleic acid linker sequence may be 35 nucleotides in length. The core promoter nucleic acid linker sequence may be 34 nucleotides in length. The core promoter nucleic acid linker sequence may be 33 nucleotides in length. The core promoter nucleic acid linker sequence may be 32 nucleotides in length. The core promoter nucleic acid linker sequence may be 31 nucleotides in length. The core promoter nucleic acid linker sequence may be 29 nucleotides in length. The core promoter nucleic acid linker sequence may be 28 nucleotides in length. The core promoter nucleic acid linker sequence may be 27 nucleotides in length. The core promoter nucleic acid linker sequence may be 26 nucleotides in length. The core promoter nucleic acid linker sequence may be 25 nucleotides in length. The core promoter nucleic acid linker sequence may be 24 nucleotides in length. The core promoter nucleic acid linker sequence may be 23 nucleotides in length. The core promoter nucleic acid linker sequence may be 22 nucleotides in length. The core promoter nucleic acid linker sequence may be 21 nucleotides in length. The core promoter nucleic acid linker sequence may be 20 nucleotides in length. The core promoter nucleic acid linker sequence may be 19 nucleotides in length. The core promoter nucleic acid linker sequence may be 18 nucleotides in length. The core promoter nucleic acid linker sequence may be 17 nucleotides in length. The core promoter nucleic acid linker sequence may be 16 nucleotides in length. The core promoter nucleic acid linker sequence may be 15 nucleotides in length. The core promoter nucleic acid linker sequence may be 14 nucleotides in length. The core promoter nucleic acid linker sequence may be 13 nucleotides in length. The core promoter nucleic acid linker sequence may be 12 nucleotides in length. The core promoter nucleic acid linker sequence may be 11 nucleotides in length. The core promoter nucleic acid linker sequence may be 10 nucleotides in length.

About 35% to about 85% of the core promoter nucleic acid linker sequence may be G or C. About 35% to about 75% of the core promoter nucleic acid linker sequence may be G or C. About 35% to about 65% of the core promoter nucleic acid linker sequence may be G or C. About 35% to about 55% of the core promoter nucleic acid linker sequence may be G or C. About 35% to about 45% of the core promoter nucleic acid linker sequence may be G or C. About 40% to about 85% of the core promoter nucleic acid linker sequence may be G or C. About 40% to about 75% of the core promoter nucleic acid linker sequence may be G or C. About 40% to about 65% of the core promoter nucleic acid linker sequence may be G or C. About 40% to about 55% of the core promoter nucleic acid linker sequence may be G or C. About 40% to about 50% of the core promoter nucleic acid linker sequence may be G or C. About 45% to about 85% of the core promoter nucleic acid linker sequence may be G or C. About 45% to about 75% of the core promoter nucleic acid linker sequence may be G or C. About 45% to about 65% of the core promoter nucleic acid linker sequence may be G or C. About 45% to about 55% of the core promoter nucleic acid linker sequence may be G or C. About 50% to about 85% of the core promoter nucleic acid linker sequence may be G or C. About 50% to about 75% of the core promoter nucleic acid linker sequence may be G or C. About 50% to about 65% of the core promoter nucleic acid linker sequence may be G or C. About 50% to about 60% of the core promoter nucleic acid linker sequence may be G or C.

About 35% of the core promoter nucleic acid linker sequence may be G or C. About 40% of the core promoter nucleic acid linker sequence may be G or C. About 45% of the core promoter nucleic acid linker sequence may be G or C. About 50% of the core promoter nucleic acid linker sequence may be G or C. About 55% of the core promoter nucleic acid linker sequence may be G or C. About 60% of the core promoter nucleic acid linker sequence may be G or C. About 65% of the core promoter nucleic acid linker sequence may be G or C. About 70% of the core promoter nucleic acid linker sequence may be G or C. About 75% of the core promoter nucleic acid linker sequence may be G or C. About 80% of the core promoter nucleic acid linker sequence may be G or C. About 85% of the core promoter nucleic acid linker sequence may be G or C.

The core promoter nucleic acid sequence may include a transcription factor binding site.

The core promoter nucleic acid linker sequence may have the sequence:

(SEQ ID NO: 1)
AGCACTGTTGGGCGTGAGTGGAGGCGCCGG,
(SEQ ID NO: 2)
CGTAGGAGTACTCGATGGTACAGATGAGCA,
(SEQ ID NO: 3)
AACGATCTACCGACTGTTTCGCAGAGGGCC,
(SEQ ID NO: 4)
CCGATAGGGTGGGCGAAGGGGCGCAGGTCC,
(SEQ ID NO: 5)
GGCCTTGGTCTGAAACTCCTGCGTCTCGCG,
(SEQ ID NO: 6)
GGTCCCTGGGTTTGCGTACTTTATCCGTCA,
(SEQ ID NO: 7)
CGCGGTGGCTCCATTAAATTGCTCCTTCCT,
(SEQ ID NO: 8)
CAATACTTGGGTCGACTTGTTATACGCGGA,
or
(SEQ ID NO: 9)
GGCGCTGCGTAAGGAGTGCTGCCAGGTGGT.

The upstream activating nucleic acid sequence may be a non-native upstream activating nucleic acid sequence (e.g. not native to a particular yeast cell). The non-native upstream activating nucleic acid sequence may be 5 to 50 nucleotides in length. The non-native upstream activating nucleic acid sequence may be 5 to 45 nucleotides in length. The non-native upstream activating nucleic acid sequence may be 5 to 40 nucleotides in length. The non-native upstream activating nucleic acid sequence may be 5 to 35 nucleotides in length. The non-native upstream activating nucleic acid sequence may be 5 to 30 nucleotides in length. The non-native upstream activating nucleic acid sequence may be 5 to 25 nucleotides in length. The non-native upstream activating nucleic acid sequence may be 5 to 20 nucleotides in length. The non-native upstream activating nucleic acid sequence may be 5 to 15 nucleotides in length. The non-native upstream activating nucleic acid sequence may be 5 to 10 nucleotides in length. The non-native upstream activating nucleic acid sequence may be 10 to 50 nucleotides in length. The non-native upstream activating nucleic acid sequence may be 10 to 45 nucleotides in length. The non-native upstream activating nucleic acid sequence may be 10 to 40 nucleotides in length. The non-native upstream activating nucleic acid sequence may be 10 to 35 nucleotides in length. The non-native upstream activating nucleic acid sequence may be 10 to 30 nucleotides in length. The non-native upstream activating nucleic acid sequence may be 10 to 25 nucleotides in length. The non-native upstream activating nucleic acid sequence may be 10 to 20 nucleotides in length. The non-native upstream activating nucleic acid sequence may be 10 to 15 nucleotides in length.

The non-native upstream activating nucleic acid sequence may be 5 nucleotides in length. The non-native upstream activating nucleic acid sequence may be 10 nucleotides in length. The non-native upstream activating nucleic acid sequence may be 11 nucleotides in length. The non-native upstream activating nucleic acid sequence may be 12 nucleotides in length. The non-native upstream activating nucleic acid sequence may be 13 nucleotides in length. The non-native upstream activating nucleic acid sequence may be 14 nucleotides in length. The non-native upstream activating nucleic acid sequence may be 15 nucleotides in length. The non-native upstream activating nucleic acid sequence may be 16 nucleotides in length. The non-native upstream activating nucleic acid sequence may be 17 nucleotides in length. The non-native upstream activating nucleic acid sequence may be 18 nucleotides in length. The non-native upstream activating nucleic acid sequence may be 19 nucleotides in length. The non-native upstream activating nucleic acid sequence may be 20 nucleotides in length. The non-native upstream activating nucleic acid sequence may be 25 nucleotides in length. The non-native upstream activating nucleic acid sequence may be 30 nucleotides in length. The non-native upstream activating nucleic acid sequence may be 25 nucleotides in length. The non-native upstream activating nucleic acid sequence may be 40 nucleotides in length. The non-native upstream activating nucleic acid sequence may be 45 nucleotides in length. The non-native upstream activating nucleic acid sequence may be 50 nucleotides in length.

The non-native upstream activating nucleic acid sequence may have the sequence: GGGGGCGGTG (SEQ ID NO:10), GCTCAACGGC (SEQ ID NO:11), TAGCATGTGA (SEQ ID NO:12), ACAGAGGGGC (SEQ ID NO:13), ACTGAAATTT (SEQ ID NO:14), or CCTCCTTGAA (SEQ ID NO:15). The non-native upstream activating nucleic acid sequence may have the sequence GGGGGCGGTG (SEQ ID NO:10). The non-native upstream activating nucleic acid sequence may have the sequence GCTCAACGGC (SEQ ID NO:11). The non-native upstream activating nucleic acid sequence may have the sequence TAGCATGTGA (SEQ ID NO:12). The non-native upstream activating nucleic acid sequence may have the sequence ACAGAGGGGC (SEQ ID NO:13). The non-native upstream activating nucleic acid sequence may have the sequence ACTGAAATTT (SEQ ID NO:14). The non-native upstream activating nucleic acid sequence may have the sequence CCTCCTTGAA (SEQ ID NO:15). The non-native upstream activating nucleic acid sequence may have the sequence: ATTGCGATGC (UASG, SEQ ID NO:35); TCCTAGCGAG (UASH, SEQ ID NO:36); TGTGCGTAAG (UASI, SEQ ID NO:37); TTTTTGAATG (UASJ, SEQ ID NO:38); GGATAGATTC (UASK, SEQ ID NO:39); TCCTAGCGAG (UASL, SEQ ID NO:40); GCCGCTTTTT (UASM, SEQ ID NO:41); TGTGCGGGTG (UASN, SEQ ID NO:42); GGGACCTTTG (UASO, SEQ ID NO:43); CCTGTATGGCGCC (UASP, SEQ ID NO:44); ACAGAGGGGC (UASQ, SEQ ID NO:45); GTTCAGGAGGCC (UASR, SEQ ID NO:46); GTTGACTCGGCC (UASS, SEQ ID NO:47); or GAGGAGGGGGCC (UAST, SEQ ID NO:48). The non-native upstream activating nucleic acid sequence may have the sequence ATTGCGATGC (SEQ ID NO:35). The non-native upstream activating nucleic acid sequence may have the sequence TCCTAGCGAG (SEQ ID NO:36). The non-native upstream activating nucleic acid sequence may have the sequence TGTGCGTAAG (SEQ ID NO:37). The non-native upstream activating nucleic acid sequence may have the sequence TTTTTGAATG (SEQ ID NO:38). The non-native upstream activating nucleic acid sequence may have the sequence GGATAGATTC (SEQ ID NO:39). The non-native upstream activating nucleic acid sequence may have the sequence TCCTAGCGAG (SEQ ID NO:40). The non-native upstream activating nucleic acid sequence may have the sequence GCCGCTTTTT (SEQ ID NO:41). The non-native upstream activating nucleic acid sequence may have the sequence TGTGCGGGTG (SEQ ID NO:42). The non-native upstream activating nucleic acid sequence may have the sequence GGGACCTTTG (SEQ ID NO:43). The non-native upstream activating nucleic acid sequence may have the sequence CCTGTATGGCGCC (SEQ ID NO:44). The non-native upstream activating nucleic acid sequence may have the sequence ACAGAGGGGC (SEQ ID NO:45). The non-native upstream activating nucleic acid sequence may have the sequence GTTCAGGAGGCC (SEQ ID NO:46). The non-native upstream activating nucleic acid sequence may have the sequence GTTGACTCGGCC (SEQ ID NO:47). The non-native upstream activating nucleic acid sequence may have the sequence GAGGAGGGGGCC (SEQ ID NO:48). The non-native upstream activating nucleic acid sequence may have the sequence CTCCGGACCACCGTCGCCCG (SEQ ID NO:49).

In embodiments, non-native upstream activating nucleic acid sequence is a plurality of non-native upstream activating nucleic acid sequences. In embodiments, the non-native upstream activating nucleic acid sequence includes at least two non-native upstream activating nucleic acid sequences. In embodiments, the non-native upstream activating nucleic acid sequence includes at least three non-native upstream activating nucleic acid sequences. In embodiments, the non-native upstream activating nucleic acid sequence includes three non-native upstream activating nucleic acid sequences. In embodiments, the non-native upstream activating nucleic acid sequence includes SEQ ID NO:12, SEQ ID NO:14 and SEQ ID NO:15. In embodiments, the non-native upstream activating nucleic acid sequence includes one or more of the non-native upstream activating nucleic acid sequences provided herein (e.g., SEQ ID NO:10-SEQ ID NO:49).

The upstream activating nucleic acid sequence may include a transcription factor binding site. The transcription factor may be a transcription factor set forth in Table 1. The transcription factor may be a Cbf1 transcription factor, a Rap1 transcription factor, a Reb1 transcription factor, a Mig1 transcription factor, a Gcn4 transcription factor, an Oaf1 transcription factor, a Rtg3 transcription factor, or a Gln3 transcription factor. The upstream activating nucleic acid sequence may be a GAL4 upstream activating sequence, a CIT upstream activating sequence, or a CLB upstream activating sequence. The upstream activating nucleic acid sequence may be a GAL4 upstream activating sequence. The upstream activating nucleic acid sequence may be a CIT upstream activating sequence. The upstream activating nucleic acid sequence may be a CLB upstream activating sequence. The upstream activating nucleic acid sequence may be a full-length GAL4 upstream activating sequence. The upstream activating nucleic acid sequence may be a full-length CIT upstream activating sequence. The upstream activating nucleic acid sequence may be a full-length CLB upstream activating sequence.

The upstream activating nucleic acid sequence may be constitutive (e.g. a constitutive-upstream activating nucleic acid sequence). The upstream activating nucleic acid sequence may be inducible (e.g. an inducible-upstream activating nucleic acid sequence). The upstream activating nucleic acid sequence may include a concatenation of two or more upstream activating nucleic acid sequences.

The upstream activating nucleic acid sequence may be repeated in tandem. When repeated in tandem, the upstream activating nucleic acid sequence may include two identical upstream activating nucleic acid sequences. Alternatively, when repeated in tandem, two different upstream activating nucleic acid sequences may be included. When repeated in tandem, the upstream activating nucleic acid sequences may be operably linked such that the tandem upstream activating nucleic acid sequences are connected with no nucleotides between the sequences. The upstream activating nucleic acid sequence may be operably linked such that a nucleotide linker (e.g. a tandem upstream activating nucleic acid sequence linker) connects the two upstream activating nucleic acid sequences.

TABLE 1
Exemplary Transcription factors (includes consensus sequences of each
transcription factor)

Abf1p
Abf2p
Aca1p
Ace2p
Adr1p
Aft1p
Aft2p
Arg80p
Arg81p
Aro80p
Arr1p
Asg1p
Ash1p
Azf1p
Bas1p
Cad1p
Cat8p
Cbf1p
Cep3p
Cha4p
Cin5p
Crz1p
Cst6p
Cup2p
Cup9p
Dal80p
Dal81p
Dal82p
Dot6p
Ecm22p
Ecm23p
Eds1p
Ert1p
Fhl1p
Fkh1p
Fkh2p
Flo8p
Fzf1p
Gal4p
Gat1p
Gat3p
Gat4p
Gcn4p
Gcr1p
Gis1p
Gln3p
Gsm1p
Gzf3p
Haa1p
Hac1p
Hal9p
Hap1p
Hap2p
Hap3p
Hap4p
Hap5p
Hcm1p
Hmlalpha2p
Hmra2p
Hsf1p
Ime1p
Ino2p
Ino4p
Ixr1p
Kar4p
Leu3p
Lys14p
Mac1p
Mal63p
Matalpha2p
Mbp1p
Mcm1p
Met31p
Met32p
Met4p
Mga1p
Mig1p
Mig2p
Mig3p
Mot2p
Mot3p
Msn1p
Msn2p
Msn4p
Mss11p
Ndt80p
Nhp10p
Nhp6ap
Nhp6bp
Nrg1p
Nrg2p
Oaf1p
Pdr1p
Pdr3p
Pdr8p
Phd1p
Pho2p
Pho4p
Pip2p
Ppr1p
Put3p
Rap1p
Rdr1p
Rds1p
Rds2p
Rds2p
Reb1p
Rei1p
Rfx1p
Rgm1p
Rgt1p
Rim101p
Rlm1p
Rme1p
Rox1p
Rph1p
Rpn4p
Rsc30p
Rsc3p
Rsf2p
Rtg1p
Rtg3p
Sfl1p
Sfp1p
Sip4p
Skn7p
Sko1p
Smp1p
Sok2p
Spt15p
Srd1p
Stb3p
Stb4p
Stb5p
Stb5p
Ste12p
Stp1p
Stp2p
Stp3p
Stp4p
Sum1p
Sut1p
Sut2p
Swi4p
Swi5p
Tbf1p
Tbs1p
Tea1p
Tec1p
Tod6p
Tos8p
Tye7p
Uga3p
Ume6p
Upc2p
Usv1p
Vhr1p
War1p
Xbp1p
YER064C
YER130C
YER184C
YGR067C
YKL222C
YLL054C
YLR278C
YML081W
YNR063W
YPR013C
YPR015C
YPR022C
YPR196W
Yap1p
Yap3p
Yap5p
Yap6p
Yap7p
Yox1p
Yrm1p
Yrr1p
Zap1p

See e.g. website: yeastract.com/consensuslist.php.

The upstream activating nucleic acid sequence may be a native upstream activating nucleic acid sequence (e.g. native to a particular yeast cell) as understood by those skilled in the art.

The tandem upstream activating nucleic acid sequence linker may be 1 to 100 nucleotides in length. The tandem upstream activating nucleic acid sequence linker may be 1 to 75 nucleotides in length. The tandem upstream activating nucleic acid sequence linker may be 1 to 50 nucleotides in length. The tandem upstream activating nucleic acid sequence linker may be 1 to 45 nucleotides in length. The tandem upstream activating nucleic acid sequence linker may be 1 to 40 nucleotides in length. The tandem upstream activating nucleic acid sequence linker may be 1 to 35 nucleotides in length. The tandem upstream activating nucleic acid sequence linker may be 1 to 30 nucleotides in length. The tandem upstream activating nucleic acid sequence linker may be 1 to 25 nucleotides in length. The tandem upstream activating nucleic acid sequence linker may be 1 to 20 nucleotides in length. The tandem upstream activating nucleic acid sequence linker may be 1 to 15 nucleotides in length. The tandem upstream activating nucleic acid sequence linker may be 1 to 10 nucleotides in length.

The tandem upstream activating nucleic acid sequence linker may be 5 nucleotides in length. The tandem upstream activating nucleic acid sequence linker may be 10 nucleotides in length. The tandem upstream activating nucleic acid sequence linker may be 15 nucleotides in length. The tandem upstream activating nucleic acid sequence linker may be 20 nucleotides in length. The tandem upstream activating nucleic acid sequence linker may be 25 nucleotides in length. The tandem upstream activating nucleic acid sequence linker may be 30 nucleotides in length. The tandem upstream activating nucleic acid sequence linker may be 35 nucleotides in length. The tandem upstream activating nucleic acid sequence linker may be 40 nucleotides in length. The tandem upstream activating nucleic acid sequence linker may be 45 nucleotides in length. The tandem upstream activating nucleic acid sequence linker may be 50 nucleotides in length. The tandem upstream activating nucleic acid sequence linker may be 55 nucleotides in length. The tandem upstream activating nucleic acid sequence linker may be 60 nucleotides in length. The tandem upstream activating nucleic acid sequence linker may be 65 nucleotides in length. The tandem upstream activating nucleic acid sequence linker may be 70 nucleotides in length. The tandem upstream activating nucleic acid sequence linker may be 75 nucleotides in length.

The two or more upstream activating nucleic acid sequence are repeated in tandem, the upstream activating nucleic acid sequences may be non-native upstream activating nucleic acid sequences, native upstream activating nucleic acid sequences or a combination thereof.

The upstream spacer nucleic acid sequence may be 5 to 55 nucleotides in length. The upstream spacer nucleic acid sequence may be 5 to 50 nucleotides in length. The upstream spacer nucleic acid sequence may be 5 to 45 nucleotides in length. The upstream spacer nucleic acid sequence may be 5 to 40 nucleotides in length. The upstream spacer nucleic acid sequence may be 5 to 35 nucleotides in length. The upstream spacer nucleic acid sequence may be 5 to 30 nucleotides in length. The upstream spacer nucleic acid sequence may be 5 to 25 nucleotides in length. The upstream spacer nucleic acid sequence may be 5 to 20 nucleotides in length. The upstream spacer nucleic acid sequence may be 5 to 15 nucleotides in length. The upstream spacer nucleic acid sequence may be 5 to 10 nucleotides in length. The upstream spacer nucleic acid sequence may be 10 to 50 nucleotides in length. The upstream spacer nucleic acid sequence may be 10 to 45 nucleotides in length. The upstream spacer nucleic acid sequence may be 10 to 40 nucleotides in length. The upstream spacer nucleic acid sequence may be 10 to 35 nucleotides in length. The upstream spacer nucleic acid sequence may be 10 to 30 nucleotides in length. The upstream spacer nucleic acid sequence may be 10 to 25 nucleotides in length. The upstream spacer nucleic acid sequence may be 10 to 20 nucleotides in length. The upstream spacer nucleic acid sequence may be 10 to 15 nucleotides in length.

The upstream spacer nucleic acid sequence may be 15 to 50 nucleotides in length. The upstream spacer nucleic acid sequence may be 15 to 45 nucleotides in length. The upstream spacer nucleic acid sequence may be 15 to 40 nucleotides in length. The upstream spacer nucleic acid sequence may be 15 to 35 nucleotides in length. The upstream spacer nucleic acid sequence may be 15 to 30 nucleotides in length. The upstream spacer nucleic acid sequence may be 15 to 25 nucleotides in length. The upstream spacer nucleic acid sequence may be 15 to 20 nucleotides in length. The upstream spacer nucleic acid sequence may be 20 to 50 nucleotides in length. The upstream spacer nucleic acid sequence may be 20 to 45 nucleotides in length. The upstream spacer nucleic acid sequence may be 20 to 40 nucleotides in length. The upstream spacer nucleic acid sequence may be 20 to 35 nucleotides in length. The upstream spacer nucleic acid sequence may be 20 to 30 nucleotides in length. The upstream spacer nucleic acid sequence may be 20 to 25 nucleotides in length.

The upstream spacer nucleic acid sequence may be 5 nucleotides in length. The upstream spacer nucleic acid sequence may be 10 nucleotides in length. The upstream spacer nucleic acid sequence may be 11 nucleotides in length. The upstream spacer nucleic acid sequence may be 12 nucleotides in length. The upstream spacer nucleic acid sequence may be 13 nucleotides in length. The upstream spacer nucleic acid sequence may be 14 nucleotides in length. The upstream spacer nucleic acid sequence may be 15 nucleotides in length. The upstream spacer nucleic acid sequence may be 16 nucleotides in length. The upstream spacer nucleic acid sequence may be 17 nucleotides in length. The upstream spacer nucleic acid sequence may be 18 nucleotides in length. The upstream spacer nucleic acid sequence may be 19 nucleotides in length. The upstream spacer nucleic acid sequence may be 20 nucleotides in length. The upstream spacer nucleic acid sequence may be 25 nucleotides in length. The upstream spacer nucleic acid sequence may be 30 nucleotides in length. The upstream spacer nucleic acid sequence may be 35 nucleotides in length. The upstream spacer nucleic acid sequence may be 40 nucleotides in length. The upstream spacer nucleic acid sequence may be 45 nucleotides in length. The upstream spacer nucleic acid sequence may be 50 nucleotides in length. The upstream spacer nucleic acid sequence may be 55 nucleotides in length.

The exogenous fungi transcription promoter nucleic acid sequences described herein may have a length of about 30 to 300 nucleotides. The exogenous fungi transcription promoter nucleic acid sequences described herein may have a length of about 30 to 250 nucleotides. The exogenous fungi transcription promoter nucleic acid sequences described herein may have a length of about 30 to 200 nucleotides. The exogenous fungi transcription promoter nucleic acid sequences described herein may have a length of about 30 to 150 nucleotides. The exogenous fungi transcription promoter nucleic acid sequences described herein may have a length of about 30 to 100 nucleotides. The exogenous fungi transcription promoter nucleic acid sequences described herein may have a length of about 30 to 50 nucleotides. The exogenous fungi transcription promoter nucleic acid sequences described herein may have a length of about 50 to 300 nucleotides. The exogenous fungi transcription promoter nucleic acid sequences described herein may have a length of about 50 to 250 nucleotides. The exogenous fungi transcription promoter nucleic acid sequences described herein may have a length of about 50 to 200 nucleotides. The exogenous fungi transcription promoter nucleic acid sequences described herein may have a length of about 50 to 150 nucleotides. The exogenous fungi transcription promoter nucleic acid sequences described herein may have a length of about 50 to 100 nucleotides. The exogenous fungi transcription promoter nucleic acid sequences described herein may have a length of about 50 to 75 nucleotides.

The exogenous fungi transcription promoter nucleic acid sequences described herein may have a length of about 30 nucleotides. The exogenous fungi transcription promoter nucleic acid sequences described herein may have a length of about 35 nucleotides. The exogenous fungi transcription promoter nucleic acid sequences described herein may have a length of about 30 nucleotides. The exogenous fungi transcription promoter nucleic acid sequences described herein may have a length of about 40 nucleotides. The exogenous fungi transcription promoter nucleic acid sequences described herein may have a length of about 45 nucleotides. The exogenous fungi transcription promoter nucleic acid sequences described herein may have a length of about 50 nucleotides. The exogenous fungi transcription promoter nucleic acid sequences described herein may have a length of about 55 nucleotides. The exogenous fungi transcription promoter nucleic acid sequences described herein may have a length of about 60 nucleotides. The exogenous fungi transcription promoter nucleic acid sequences described herein may have a length of about 65 nucleotides. The exogenous fungi transcription promoter nucleic acid sequences described herein may have a length of about 70 nucleotides. The exogenous fungi transcription promoter nucleic acid sequences described herein may have a length of about 75 nucleotides. The exogenous fungi transcription promoter nucleic acid sequences described herein may have a length of about 80 nucleotides. The exogenous fungi transcription promoter nucleic acid sequences described herein may have a length of about 85 nucleotides. The exogenous fungi transcription promoter nucleic acid sequences described herein may have a length of about 90 nucleotides. The exogenous fungi transcription promoter nucleic acid sequences described herein may have a length of about 95 nucleotides. The exogenous fungi transcription promoter nucleic acid sequences described herein may have a length of about 100 nucleotides.

The exogenous fungi transcription promoter nucleic acid sequences described herein may have a length of about 110 nucleotides. The exogenous fungi transcription promoter nucleic acid sequences described herein may have a length of about 120 nucleotides. The exogenous fungi transcription promoter nucleic acid sequences described herein may have a length of about 130 nucleotides. The exogenous fungi transcription promoter nucleic acid sequences described herein may have a length of about 140 nucleotides. The exogenous fungi transcription promoter nucleic acid sequences described herein may have a length of about 150 nucleotides. The exogenous fungi transcription promoter nucleic acid sequences described herein may have a length of about 160 nucleotides. The exogenous fungi transcription promoter nucleic acid sequences described herein may have a length of about 170 nucleotides. The exogenous fungi transcription promoter nucleic acid sequences described herein may have a length of about 180 nucleotides. The exogenous fungi transcription promoter nucleic acid sequences described herein may have a length of about 190 nucleotides. The exogenous fungi transcription promoter nucleic acid sequences described herein may have a length of about 200 nucleotides. The exogenous fungi transcription promoter nucleic acid sequences described herein may have a length of about 225 nucleotides. The exogenous fungi transcription promoter nucleic acid sequences described herein may have a length of about 250 nucleotides. The exogenous fungi transcription promoter nucleic acid sequences described herein may have a length of about 275 nucleotides. The exogenous fungi transcription promoter nucleic acid sequences described herein may have a length of about 300 nucleotides.

### II. EXPRESSION CONSTRUCTS

Also provided herein are expression constructs which include an exogenous fungi transcription promoter nucleic acid sequence described herein. The expression construct may be a plasmid. The expression construct may be a genome. The expression construct may be an artificial chromosome (e.g. a yeast artificial chromosome (YAC)). The exogenous fungi transcription promoter nucleic acid sequence may be operably linked to a 5′ open reading frame of a gene. The gene may be a native gene (i.e. a gene or gene product naturally found (endogenously) in the host). The gene may be a non-native gene (i.e. a heterologous gene or gene product not naturally found in the host). The exogenous fungi transcription promoter nucleic acid sequence may increase the expression of the gene in the expression construct when compared to a control (e.g. expression using a native promoter sequence (e.g. a native CYC1 promoter)). The exogenous fungi transcription promoter nucleic acid sequence may decrease the expression of the gene in the expression construct when compared to a control (e.g. expression using a native promoter sequence (e.g. a native CYC1 promoter)).

The expression construct may contain one or more exogenous fungi transcription promoter nucleic acid sequences, which may be the same for each gene in the construct. The expression construct may contain one or more exogenous fungi transcription promoter nucleic acid sequences, which may optionally be the different for each gene in the construct. The different exogenous transcription promoter nucleic acid sequences may allow for independent control of the level of expression of each gene. Thus, in such embodiments, each independent exogenous transcription promoter nucleic acid sequence in an expression construct may independently modulate the expression of the gene to which it is operably linked.

### III. FUNGI CELLS

Provided herein is a fungi cell that includes an exogenous transcription promoter nucleic acid sequence. The fungi cell may be a yeast cell. The yeast cell may be a Saccharomyces cerevisiae yeast cell, a Yarrowia lipolytica yeast cell, a Candida intermedia yeast cell, a Cryptococcos neoformans yeast cell, a Debaryomyces hansenii yeast cell, a Kluyveromyces lactis yeast cell, a Torulaspora delbrueckii yeast cell, a Zygosaccharomyces rouxii yeast cell, a Phaffia rhodozyma yeast cell, or a Scheffersomyces stipitis yeast cell. The yeast cell may be a Saccharomyces cerevisiae yeast cell or a Yarrowia lipolytica yeast cell. The yeast cell may be a Saccharomyces cerevisiae yeast cell. The yeast cell may be a Yarrowia lipolytica yeast cell. The yeast cell may be a Candida intermedia yeast cell. The yeast cell may be a Cryptococcos neoformans yeast cell. The yeast cell may be a Debaryomyces hansenii yeast cell. The yeast cell may be a Phaffia rhodozyma yeast cell. The yeast cell may be a Scheffersomyces stipitis yeast cell. The yeast cell may be a Kluyveromyces lactis yeast cell. The yeast cell may be a Torulaspora delbrueckii yeast cell. The yeast cell may be a Zygosaccharomyces rouxii yeast cell. The exogenous fungi transcription promoter nucleic acid sequence may be located on an expression construct as described herein.

The exogenous fungi transcription promoter nucleic acid sequence may be 5′ operably linked to an open reading frame (ORF) of a gene in the fungi cell. The gene may be an endogenous gene in the host cell (e.g. yeast cell). The exogenous fungi transcription promoter nucleic acid sequence may be 5′ operably linked to an ORF where the sequence is operably linked to a gene in a host cell (e.g. a yeast cell) through a recombination event. The gene may be a heterologous gene (i.e. a non-native gene). In such embodiments, the exogenous fungi transcription promoter nucleic acid sequence is expressed heterologously in the fungi cell. The gene may be on the fungi cell chromosome (through, for example, a recombination event such as homologous recombination) or on an expression construction (i.e. a plasmid or a yeast artificial chromosome (YAC)).

The exogenous fungi transcription promoter nucleic acid sequence may increase expression of a gene (e.g. an endogenous or heterologous gene) in the fungi cell compared to a control (e.g. absence of the exogenous fungi transcription promoter nucleic acid sequence or expression using a native promoter sequence (e.g. a native CYC1 promoter)). The exogenous fungi transcription promoter nucleic acid sequence may decrease expression of a gene (e.g. an endogenous or heterologous gene) in the fungi cell compared to a control (e.g. absence of the exogenous fungi transcription promoter nucleic acid sequence or expression using a native promoter sequence (e.g. a native CYC1 promoter)). The sequence of the exogenous fungi transcription promoter nucleic acid sequence may prevent or reduce homologous recombination of the exogenous fungi transcription promoter nucleic acid sequence into a host cell (e.g. a yeast cell) chromosome.

### IV. METHODS OF EXPRESSION

Provided herein are methods of expressing a gene in a fungi cell. In one aspect is a method of expressing a gene in a fungi cell by transforming the fungi cell with an expression construct described herein that includes a gene operably linked to an exogenous fungi transcription promoter nucleic acid sequence described herein. The cell is allowed to express the expression construct, and the exogenous fungi transcription promoter nucleic acid sequence modulates a level of transcription initiation or a rate of transcription of the gene, thereby expressing the gene in the fungi cell. In embodiments, a fungi cell is transformed using an exogenous fungi transcription promoter nucleic acid sequence described herein, where the exogenous fungi transcription promoter nucleic acid sequence is inserted into the fungi cell genome by a recombination event (e.g. homologous recombination). The recombination event can include genome editing and use of zinc finger nucleases as understood in the art. See Dicarlo J., et. al., Nucleic Acids Research, 2013, 1-8. The gene may be an endogenous yeast gene. The gene may be a heterologous gene.

The exogenous fungi transcription promoter nucleic acid sequence may increase the level of transcription initiation or rate of transcription of the gene compared to a control (e.g. absence of the exogenous fungi transcription promoter nucleic acid sequence or expression using a native promoter sequence (e.g. a native CYC1 promoter)). The exogenous fungi transcription promoter nucleic acid sequence may increase the level of transcription initiation or the rate of transcription of the gene compared to a control (e.g. absence of the exogenous fungi transcription promoter nucleic acid sequence or expression using a native promoter sequence (e.g. a native CYC1 promoter)). The exogenous fungi transcription promoter nucleic acid sequence may increase the rate of transcription of the gene compared to a control (e.g. absence of the exogenous fungi transcription promoter nucleic acid sequence or expression using a native promoter sequence (e.g. a native CYC1 promoter)). The exogenous fungi transcription promoter nucleic acid sequence may decrease the level of transcription initiation or rate of transcription of the gene when compared to a control (e.g. absence of the exogenous fungi transcription promoter nucleic acid sequence or expression using a native promoter sequence (e.g. a native CYC1 promoter)). The exogenous fungi transcription promoter nucleic acid sequence may decrease the level of transcription of the gene when compared to a control (e.g. absence of the exogenous fungi transcription promoter nucleic acid sequence or expression using a native promoter sequence (e.g. a native CYC1 promoter)). The exogenous fungi transcription promoter nucleic acid sequence may decrease the rate of transcription of the gene when compared to a control (e.g. absence of the exogenous fungi transcription promoter nucleic acid sequence or expression using a native promoter sequence (e.g. a native CYC1 promoter)).

### V. METHODS OF TESTING

Further provided herein are methods of testing fungi core promoter nucleic acid sequences. The methods are useful to identify fungi core promoter nucleic acid sequences that can initiate transcription or modulate a rate of transcription. In one aspect is a method of testing a fungi core promoter nucleic acid test sequence, by determining a level of transcription initiation or a rate of transcription of a core promoter nucleic acid test sequence. The method may be a method of testing by determining a level of transcription initiation of the core promoter nucleic acid test sequence. The method may be a method of testing by determining a rate of transcription of the core promoter nucleic acid test sequence. The core promoter nucleic acid test sequence includes a fungi TATA box sequence motif, a fungi transcription start site nucleic acid sequence, and a core promoter nucleic acid linker test sequence.

The method may further include determining a level of transcription initiation or a rate of transcription of a second core promoter nucleic acid test sequence, where the second core promoter nucleic acid test sequence includes a fungi TATA box sequence motif, a fungi transcription start site nucleic acid sequence, and a second core promoter nucleic acid linker test sequence. The second core promoter nucleic acid linker test sequence is derived from the core promoter nucleic acid linker test sequence. The core promoter nucleic acid test sequence and the second core promoter nucleic acid test sequence may have the same fungi TATA box sequence motif and the same fungi transcription start site nucleic acid sequence. The core promoter nucleic acid test sequence and the second core promoter nucleic acid test sequence may have different fungi TATA box sequence motifs or different fungi transcription start site nucleic acid sequences.

The core promoter nucleic acid test sequence may have a level of transcription initiation or a rate of transcription greater than a level of transcription initiation or a rate of transcription from a control promoter sequence. Depending on the expression conditions desired, the core promoter nucleic acid test sequence may have a level of transcription initiation or a rate of transcription less than a level of transcription initiation or a rate of transcription from a control promoter sequence. Thus, a core promoter nucleic acid test sequence can be selected for its level of transcription initiation or rate of transcription and its modulation of the expression of a gene to which it may be 5′ operably linked. The control promoter sequence may be a native yeast promoter. The native yeast promoter may be a native promoter. The native promoter may be a TEF1 promoter, TEF2 promoter, ADH1 promoter, TDH3 promoter, CLB1 promoter, STE5 promoter, PGI1 promoter, TPI1 promoter, FBA1 promoter, PDC1 promoter, ENO2 promoter, CYC1 promoter. The native promoter may be a CYC1 promoter. The control may be a level of transcription initiation or a rate of transcription from another core promoter sequence having a different sequence from the core promoter nucleic acid test sequence or the second core promoter nucleic acid test sequence.

Likewise, the second core promoter nucleic acid test sequence may have a level of transcription initiation or a rate of transcription greater than a level of transcription initiation or a rate of transcription from a control promoter sequence. The second core promoter nucleic acid test sequence may have a level of transcription initiation or a rate of transcription greater than a level of transcription initiation or a rate of transcription from the core promoter nucleic acid test sequence. The second core promoter nucleic acid test sequence may have a level of transcription initiation or a rate of transcription less than a level of transcription initiation or rate of transcription from a control promoter sequence or less than a level of transcription initiation or a rate of transcription from the core promoter nucleic acid test sequence. A second core promoter nucleic acid test sequence may therefore be selected for its level of transcription initiation or rate of transcription and its modulation of the expression of a gene to which it may be 5′ operably linked. The control promoter sequence may be a native yeast promoter described herein. The native yeast promoter may be a CYC1 promoter. The control may be a level of transcription initiation or a rate of transcription from another core promoter sequence having a different sequence from the core promoter nucleic acid test sequence or the second core promoter nucleic acid test sequence.

The sequence of the core promoter nucleic acid test sequence or second core promoter nucleic acid test sequence may be determined. The sequence of the core promoter nucleic acid test sequence or second core promoter nucleic acid test sequence may be determined using nucleic acid sequencing techniques known in the art.

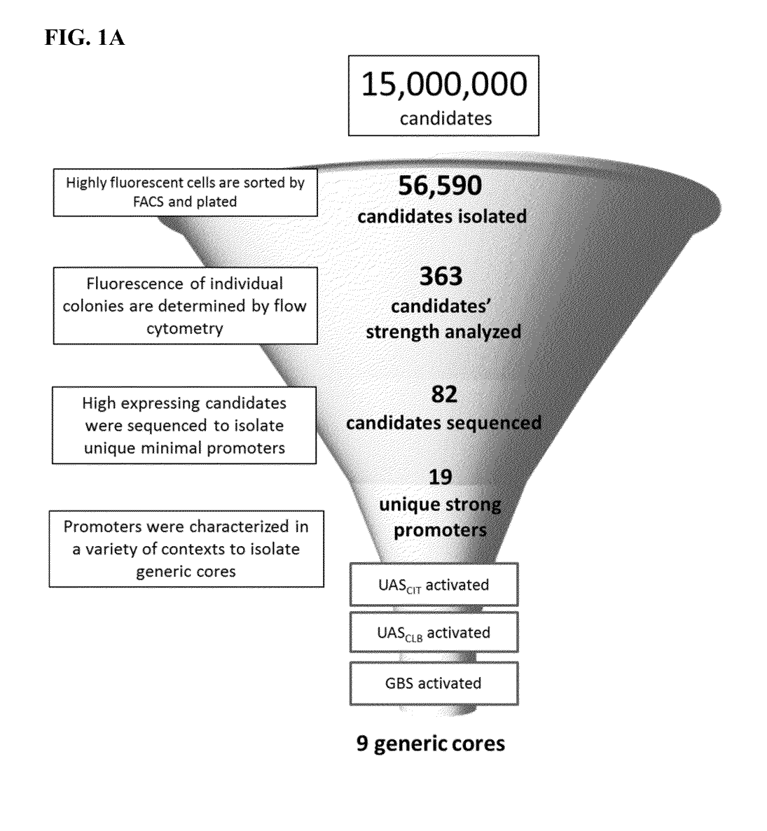

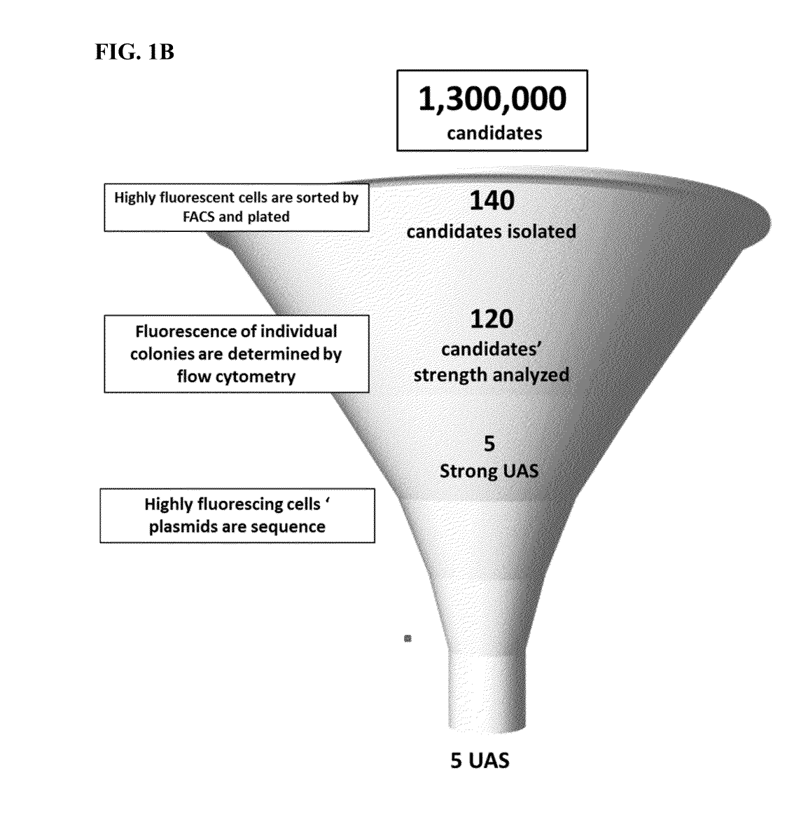

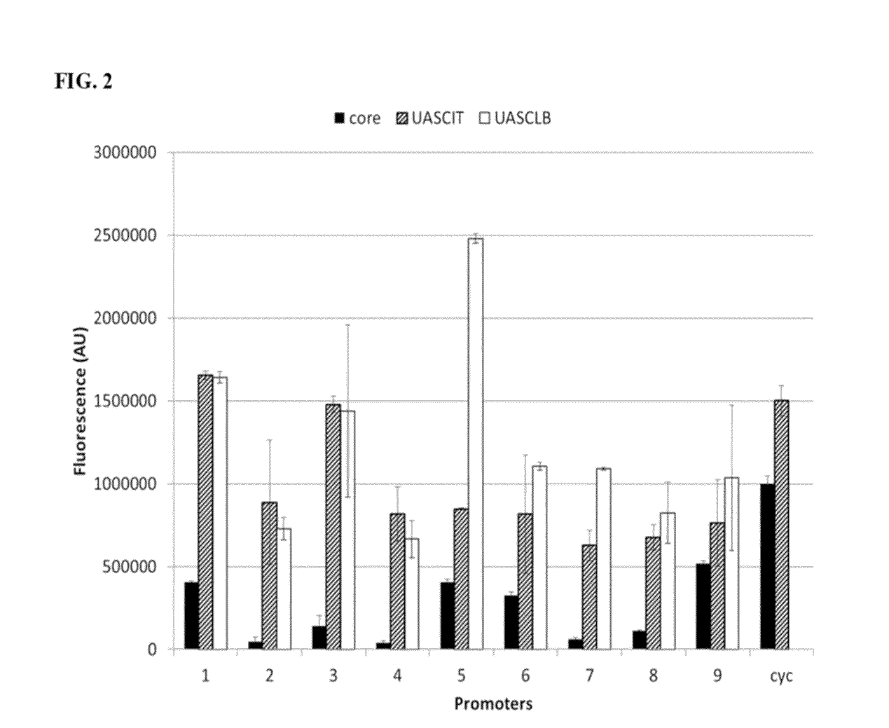

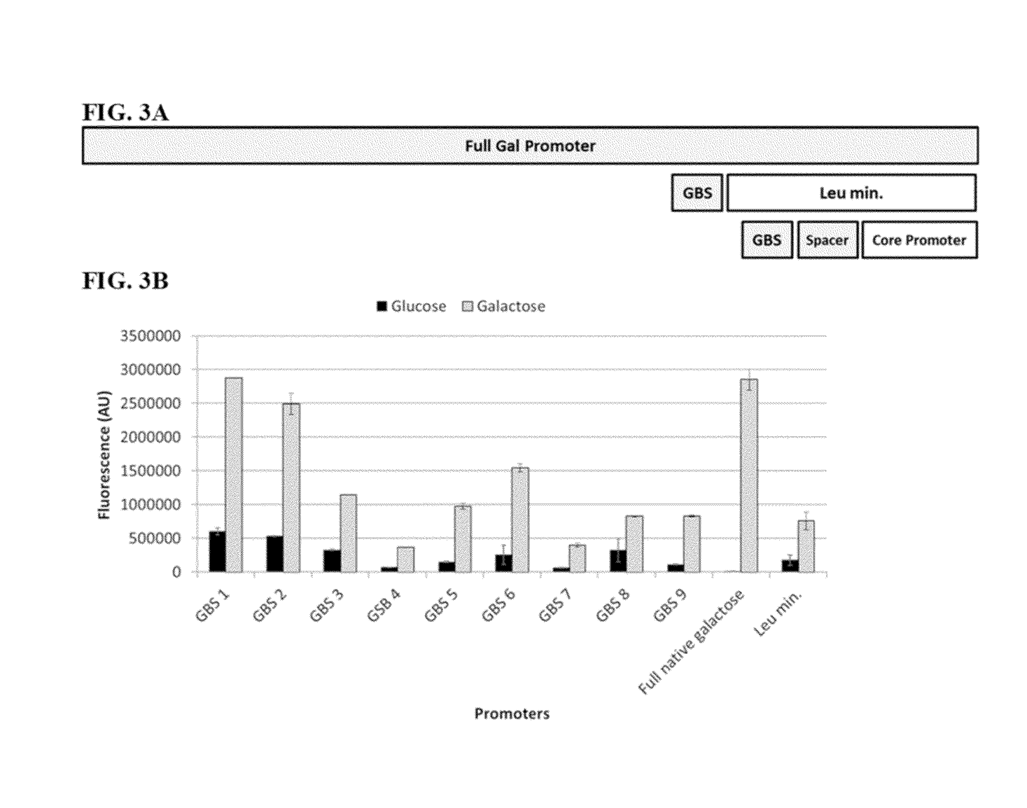

The core promoter nucleic acid test sequence or second core promoter nucleic acid test sequence may be included in a plurality of core promoter nucleic acid test sequences (e.g. a library). The library may be synthesized using known techniques in the art. Thus, the core promoter nucleic acid test sequence may be identified in one or more rounds of testing of core promoter nucleic acid test sequences for transcription initiation or rate of transcription and consistent expression under multiple contexts as exemplified by FIGS. 1A-1B. The second core promoter nucleic acid test sequence may be identified from such a library or may be derived from one of the plurality of core promoter nucleic acid test sequences. When derived from a core promoter nucleic acid test sequence, the second core promoter nucleic acid test sequence may include the same fungi TATA box sequence motif and the same fungi transcription start site nucleic acid sequence as the core promoter nucleic acid test sequence from which it is derived. When derived from one of the plurality of core promoter nucleic acid test sequences, the second core promoter nucleic acid test sequence may include a different fungi TATA box sequence motif or a different fungi transcription start site nucleic acid sequence as the core promoter nucleic acid test sequence from which it was derived.

The fungi TATA box sequence motif and a fungi transcription start site nucleic acid sequence of the core promoter nucleic acid test sequence and second core promoter nucleic acid test sequence are as described hereinabove in section I.

Detecting the level of transcription initiation or rate of transcription may be performed using techniques known in the art. The level of transcription initiation or rate of transcription may be detected using fluorescence or an enzymatic activity assay. The core promoter nucleic acid test sequence or second core promoter nucleic acid test sequence may include a detectable moiety. The detectable moiety may be measured to determine the level of transcription initiation or the rate of transcription by the test sequence. The detectable moiety may be a protein translated from RNA transcribed from transcription of the gene operably linked to the core promoter nucleic acid test sequence or to the second core promoter nucleic acid test sequence. The detectable moiety may be a RNA transcribed from the gene operably linked to the core promoter nucleic acid test sequence or to the second core promoter nucleic acid test sequence.

The core promoter nucleic acid linker test sequence and second core promoter nucleic acid linker test sequences may independently be 5 to 55 nucleotides in length. The core promoter nucleic acid linker test sequence and second core promoter nucleic acid linker test sequences may independently be 5 to 50 nucleotides in length. The core promoter nucleic acid linker test sequence and second core promoter nucleic acid linker test sequences may independently be 5 to 40 nucleotides in length. The core promoter nucleic acid linker test sequence and second core promoter nucleic acid linker test sequences may independently be 5 to 35 nucleotides in length. The core promoter nucleic acid linker test sequence and second core promoter nucleic acid linker test sequences may independently be 5 to 30 nucleotides in length. The core promoter nucleic acid linker test sequence and second core promoter nucleic acid linker test sequences may independently be 5 to 25 nucleotides in length. The core promoter nucleic acid linker test sequence and second core promoter nucleic acid linker test sequences may independently be 5 to 20 nucleotides in length. The core promoter nucleic acid linker test sequence and second core promoter nucleic acid linker test sequences may independently be 5 to 15 nucleotides in length. The core promoter nucleic acid linker test sequence and second core promoter nucleic acid linker test sequences may independently be 5 to 10 nucleotides in length.

The core promoter nucleic acid linker test sequence and second core promoter nucleic acid linker test sequences may independently be 10 to 55 nucleotides in length. The core promoter nucleic acid linker test sequence and second core promoter nucleic acid linker test sequences may independently be 10 to 50 nucleotides in length. The core promoter nucleic acid linker test sequence and second core promoter nucleic acid linker test sequences may independently be 10 to 45 nucleotides in length. The core promoter nucleic acid linker test sequence and second core promoter nucleic acid linker test sequences may independently be 10 to 40 nucleotides in length. The core promoter nucleic acid linker test sequence and second core promoter nucleic acid linker test sequences may independently be 10 to 35 nucleotides in length. The core promoter nucleic acid linker test sequence and second core promoter nucleic acid linker test sequences may independently be 10 to 30 nucleotides in length. The core promoter nucleic acid linker test sequence and second core promoter nucleic acid linker test sequences may independently be 10 to 25 nucleotides in length. The core promoter nucleic acid linker test sequence and second core promoter nucleic acid linker test sequences may independently be 10 to 20 nucleotides in length. The core promoter nucleic acid linker test sequence and second core promoter nucleic acid linker test sequences may independently be 10 to 15 nucleotides in length.

The core promoter nucleic acid linker test sequence and second core promoter nucleic acid linker test sequences may independently be 15 to 55 nucleotides in length. The core promoter nucleic acid linker test sequence and second core promoter nucleic acid linker test sequences may independently be 15 to 50 nucleotides in length. The core promoter nucleic acid linker test sequence and second core promoter nucleic acid linker test sequences may independently be 15 to 45 nucleotides in length. The core promoter nucleic acid linker test sequence and second core promoter nucleic acid linker test sequences may independently be 15 to 40 nucleotides in length. The core promoter nucleic acid linker test sequence and second core promoter nucleic acid linker test sequences may independently be 15 to 35 nucleotides in length. The core promoter nucleic acid linker test sequence and second core promoter nucleic acid linker test sequences may independently be 15 to 30 nucleotides in length. The core promoter nucleic acid linker test sequence and second core promoter nucleic acid linker test sequences may independently be 15 to 25 nucleotides in length. The core promoter nucleic acid linker test sequence and second core promoter nucleic acid linker test sequences may independently be 15 to 20 nucleotides in length.

The core promoter nucleic acid linker test sequence and second core promoter nucleic acid linker test sequences may independently be 5 nucleotides in length. The core promoter nucleic acid linker test sequence and second core promoter nucleic acid linker test sequences may independently be 6 nucleotides in length. The core promoter nucleic acid linker test sequence and second core promoter nucleic acid linker test sequences may independently be 7 nucleotides in length. The core promoter nucleic acid linker test sequence and second core promoter nucleic acid linker test sequences may independently be 8 nucleotides in length. The core promoter nucleic acid linker test sequence and second core promoter nucleic acid linker test sequences may independently be 9 nucleotides in length. The core promoter nucleic acid linker test sequence and second core promoter nucleic acid linker test sequences may independently be 10 nucleotides in length. The core promoter nucleic acid linker test sequence and second core promoter nucleic acid linker test sequences may independently be 11 nucleotides in length. The core promoter nucleic acid linker test sequence and second core promoter nucleic acid linker test sequences may independently be 12 nucleotides in length. The core promoter nucleic acid linker test sequence and second core promoter nucleic acid linker test sequences may independently be 13 nucleotides in length. The core promoter nucleic acid linker test sequence and second core promoter nucleic acid linker test sequences may independently be 14 nucleotides in length. The core promoter nucleic acid linker test sequence and second core promoter nucleic acid linker test sequences may independently be 15 nucleotides in length. The core promoter nucleic acid linker test sequence and second core promoter nucleic acid linker test sequences may independently be 16 nucleotides in length. The core promoter nucleic acid linker test sequence and second core promoter nucleic acid linker test sequences may independently be 17 nucleotides in length. The core promoter nucleic acid linker test sequence and second core promoter nucleic acid linker test sequences may independently be 18 nucleotides in length. The core promoter nucleic acid linker test sequence and second core promoter nucleic acid linker test sequences may independently be 19 nucleotides in length. The core promoter nucleic acid linker test sequence and second core promoter nucleic acid linker test sequences may independently be 20 nucleotides in length. The core promoter nucleic acid linker test sequence and second core promoter nucleic acid linker test sequences may independently be 21 nucleotides in length. The core promoter nucleic acid linker test sequence and second core promoter nucleic acid linker test sequences may independently be 22 nucleotides in length. The core promoter nucleic acid linker test sequence and second core promoter nucleic acid linker test sequences may independently be 23 nucleotides in length. The core promoter nucleic acid linker test sequence and second core promoter nucleic acid linker test sequences may independently be 24 nucleotides in length. The core promoter nucleic acid linker test sequence and second core promoter nucleic acid linker test sequences may independently be 25 nucleotides in length. The core promoter nucleic acid linker test sequence and second core promoter nucleic acid linker test sequences may independently be 26 nucleotides in length. The core promoter nucleic acid linker test sequence and second core promoter nucleic acid linker test sequences may independently be 27 nucleotides in length. The core promoter nucleic acid linker test sequence and second core promoter nucleic acid linker test sequences may independently be 28 nucleotides in length. The core promoter nucleic acid linker test sequence and second core promoter nucleic acid linker test sequences may independently be 29 nucleotides in length. The core promoter nucleic acid linker test sequence and second core promoter nucleic acid linker test sequences may independently be 30 nucleotides in length.

The core promoter nucleic acid linker test sequence and second core promoter nucleic acid linker test sequences may independently be 35 nucleotides in length. The core promoter nucleic acid linker test sequence and second core promoter nucleic acid linker test sequences may independently be 40 nucleotides in length. The core promoter nucleic acid linker test sequence and second core promoter nucleic acid linker test sequences may independently be 45 nucleotides in length. The core promoter nucleic acid linker test sequence and second core promoter nucleic acid linker test sequences may independently be 50 nucleotides in length. The core promoter nucleic acid linker test sequence and second core promoter nucleic acid linker test sequences may independently be 55 nucleotides in length. The core promoter nucleic acid linker test sequence and second core promoter nucleic acid linker test sequences may independently be 5 nucleotides in length. The core promoter nucleic acid linker test sequence and second core promoter nucleic acid linker test sequence may independently be 15, 18, 20, 21, 24, 25, 27, or 30 nucleotides in length.

The core promoter nucleic acid test sequence may further include an upstream activating nucleic acid sequence 5′ to the fungi TATA box sequence motif. The core promoter nucleic acid test sequence and the upstream activating nucleic acid sequence may be linked by an upstream spacer nucleic acid test sequence. The upstream activating nucleic acid sequence is as described herein.

The upstream spacer nucleic acid test sequence may be 5 to 50 nucleotides in length. The upstream spacer nucleic acid test sequence may be 5 to 45 nucleotides in length. The upstream spacer nucleic acid test sequence may be 5 to 40 nucleotides in length. The upstream spacer nucleic acid test sequence may be 5 to 35 nucleotides in length. The upstream spacer nucleic acid test sequence may be 5 to 30 nucleotides in length. The upstream spacer nucleic acid test sequence may be 5 to 25 nucleotides in length. The upstream spacer nucleic acid test sequence may be 5 to 20 nucleotides in length. The upstream spacer nucleic acid test sequence may be 5 to 15 nucleotides in length. The upstream spacer nucleic acid test sequence may be 5 to 10 nucleotides in length.

The upstream spacer nucleic acid test sequence may be 10 to 50 nucleotides in length. The upstream spacer nucleic acid test sequence may be 10 to 45 nucleotides in length. The upstream spacer nucleic acid test sequence may be 10 to 40 nucleotides in length. The upstream spacer nucleic acid test sequence may be 10 to 35 nucleotides in length. The upstream spacer nucleic acid test sequence may be 10 to 30 nucleotides in length. The upstream spacer nucleic acid test sequence may be 10 to 25 nucleotides in length. The upstream spacer nucleic acid test sequence may be 10 to 20 nucleotides in length. The upstream spacer nucleic acid test sequence may be 10 to 15 nucleotides in length.

The upstream spacer nucleic acid test sequence may be 15 to 50 nucleotides in length. The upstream spacer nucleic acid test sequence may be 15 to 45 nucleotides in length. The upstream spacer nucleic acid test sequence may be 15 to 40 nucleotides in length. The upstream spacer nucleic acid test sequence may be 15 to 35 nucleotides in length. The upstream spacer nucleic acid test sequence may be 15 to 30 nucleotides in length. The upstream spacer nucleic acid test sequence may be 15 to 25 nucleotides in length. The upstream spacer nucleic acid test sequence may be 15 to 20 nucleotides in length.

The upstream spacer nucleic acid test sequence may be 5 nucleotides in length. The upstream spacer nucleic acid test sequence may be 10 nucleotides in length. The upstream spacer nucleic acid test sequence may be 11 nucleotides in length. The upstream spacer nucleic acid test sequence may be 12 nucleotides in length. The upstream spacer nucleic acid test sequence may be 13 nucleotides in length. The upstream spacer nucleic acid test sequence may be 14 nucleotides in length. The upstream spacer nucleic acid test sequence may be 15 nucleotides in length. The upstream spacer nucleic acid test sequence may be 16 nucleotides in length. The upstream spacer nucleic acid test sequence may be 17 nucleotides in length. The upstream spacer nucleic acid test sequence may be 18 nucleotides in length. The upstream spacer nucleic acid test sequence may be 19 nucleotides in length. The upstream spacer nucleic acid test sequence may be 20 nucleotides in length. The upstream spacer nucleic acid test sequence may be 21 nucleotides in length. The upstream spacer nucleic acid test sequence may be 22 nucleotides in length. The upstream spacer nucleic acid test sequence may be 23 nucleotides in length. The upstream spacer nucleic acid test sequence may be 24 nucleotides in length. The upstream spacer nucleic acid test sequence may be 25 nucleotides in length. The upstream spacer nucleic acid test sequence may be 26 nucleotides in length. The upstream spacer nucleic acid test sequence may be 27 nucleotides in length. The upstream spacer nucleic acid test sequence may be 28 nucleotides in length. The upstream spacer nucleic acid test sequence may be 29 nucleotides in length. The upstream spacer nucleic acid test sequence may be 30 nucleotides in length. The upstream spacer nucleic acid test sequence may be 31 nucleotides in length. The upstream spacer nucleic acid test sequence may be 32 nucleotides in length. The upstream spacer nucleic acid test sequence may be 33 nucleotides in length. The upstream spacer nucleic acid test sequence may be 34 nucleotides in length. The upstream spacer nucleic acid test sequence may be 35 nucleotides in length. The upstream spacer nucleic acid test sequence may be 36 nucleotides in length. The upstream spacer nucleic acid test sequence may be 37 nucleotides in length. The upstream spacer nucleic acid test sequence may be 38 nucleotides in length. The upstream spacer nucleic acid test sequence may be 39 nucleotides in length. The upstream spacer nucleic acid test sequence may be 40 nucleotides in length. The upstream spacer nucleic acid test sequence may be 45 nucleotides in length. The upstream spacer nucleic acid test sequence may be 50 nucleotides in length.

Also provided herein are methods for testing an upstream activating nucleic acid sequence. In one aspect is a method of testing an upstream activating nucleic acid sequence by determining a level of transcription initiation or a rate of transcription of a fungi transcription promoter nucleic acid test sequence comprising a non-native upstream activating nucleic acid test sequence, a fungi promoter sequence, and an upstream spacer nucleic acid test sequence which links the non-native upstream activating nucleic acid test sequence and the fungi promoter sequence. As a control, the level of transcription initiation or rate of transcription of a fungi transcription promoter nucleic acid test sequence may be determined in the absence of the upstream activating nucleic acid sequence. Thus, the level of transcription initiation or rate of transcription attributable to a fungi transcription promoter nucleic acid test sequence may be compared to a level of transcription initiation or rate of transcription of the fungi transcription promoter nucleic acid test sequence attributable to the addition of an upstream activating nucleic acid sequence.

The method may further include determining a level of transcription initiation or a rate of transcription of a second fungi transcription promoter nucleic acid test sequence where the second fungi transcription promoter nucleic acid test sequence includes the same non-native upstream activating nucleic acid test sequence, a fungi promoter sequence, and a second upstream spacer nucleic acid test sequence. The second upstream spacer nucleic acid test sequence is derived from the upstream spacer nucleic acid test sequence. The fungi promoter sequence of the second fungi transcription promoter nucleic acid test sequence may be the same fungi promoter sequence found in the fungi transcription promoter nucleic acid test sequence.

The method may further include determining a level of transcription initiation or a rate of transcription of a second fungi transcription promoter nucleic acid test sequence where the second fungi transcription promoter nucleic acid test sequence includes a second non-native upstream activating nucleic acid test sequence, a fungi promoter sequence, and the same upstream spacer nucleic acid test sequence. The second non-native upstream activating nucleic acid test sequence is derived from the non-native upstream activating nucleic acid test sequence. The fungi promoter sequence of the second fungi transcription promoter nucleic acid test sequence may be the same fungi promoter sequence found in the fungi transcription promoter nucleic acid test sequence.

The method may further include determining a level of transcription initiation or a rate of transcription of a second fungi transcription promoter nucleic acid test sequence where the second fungi transcription promoter nucleic acid test sequence includes a second non-native upstream activating nucleic acid test sequence, a fungi promoter sequence, and a second upstream spacer nucleic acid test sequence. The second non-native upstream activating nucleic acid test sequence is derived from the non-native upstream activating nucleic acid test sequence. The second upstream spacer nucleic acid test sequence is derived from the upstream spacer nucleic acid test sequence. The fungi promoter sequence of the second fungi transcription promoter nucleic acid test sequence may be the same fungi promoter sequence found in the fungi transcription promoter nucleic acid test sequence.

The fungi transcription promoter nucleic acid test sequence may have a level of transcription initiation or a rate of transcription greater than a level of transcription initiation or a rate of transcription from a control promoter sequence. Depending on the expression conditions desired, the fungi transcription promoter nucleic acid test sequence may have a level of transcription initiation or a rate of transcription less than a level of transcription initiation or a rate of transcription from a control promoter sequence. Thus, a fungi transcription promoter nucleic acid test sequence can be selected for its level of transcription initiation or rate of transcription and its modulation of the expression of a gene to which it may be 5′ operably linked. The control promoter sequence may be a native yeast promoter. The native yeast promoter may be a CYC1 promoter. The control may be a level of transcription initiation or a rate of transcription from another fungi transcription promoter nucleic acid test sequence having a different sequence from the fungi transcription promoter nucleic acid test sequence or the second fungi transcription promoter nucleic acid test sequence.

Likewise, the second fungi transcription promoter nucleic acid test sequence may have a level of transcription initiation or a rate of transcription greater than a level of transcription initiation or rate of transcription from a control promoter sequence. The second fungi transcription promoter nucleic acid test sequence may have a level of transcription initiation or a rate of transcription greater than a level of transcription initiation or rate of transcription of the fungi transcription promoter nucleic acid test sequence. The second fungi transcription promoter nucleic acid test sequence may have a level of transcription initiation or a rate of transcription less than a level of transcription initiation or a rate of transcription from a control promoter sequence or less than a level of transcription initiation or a rate of transcription from the fungi transcription promoter nucleic acid test sequence. A second fungi transcription promoter nucleic acid test sequence may therefore be selected for its level of transcription initiation or rate of transcription and its modulation of the expression of a gene to which it may be 5′ operably linked. The control promoter sequence may be a native yeast promoter. The native yeast promoter may be a CYC1 promoter. The control may be a level of transcription initiation or a rate of transcription from another fungi transcription promoter nucleic acid test sequence having a different sequence from the fungi transcription promoter nucleic acid test sequence or the second fungi transcription promoter nucleic acid test sequence.

The sequence of the fungi transcription promoter nucleic acid test sequence or second fungi transcription promoter nucleic acid test sequence may be determined. The sequence of the fungi transcription promoter nucleic acid test sequence or second fungi transcription promoter nucleic acid test sequence may be determined using nucleic acid sequencing techniques known in the art.

The fungi transcription promoter nucleic acid test sequence or second fungi transcription promoter nucleic acid test sequence may be included in a plurality of fungi transcription promoter nucleic acid test sequences (e.g. a library). The library may be synthesized using known techniques in the art. Thus, the fungi transcription promoter nucleic acid test sequence may be identified in one or more rounds of testing of fungi transcription promoter nucleic acid test sequences for transcription initiation or rate of transcription. The second fungi transcription promoter nucleic acid test sequence may be identified from such a library or may be derived from one of the plurality of the fungi transcription promoter nucleic acid test sequences.

The fungi promoter sequence may be a native-fungi promoter sequence (e.g. a CYC1 promoter nucleic acid sequence). The fungi promoter sequence may be a core promoter nucleic acid sequence described herein.

Detecting the level of transcription initiation or rate of transcription may be performed using techniques known in the art. The level of transcription initiation or rate of transcription may be detected using fluorescence. The fungi transcription promoter nucleic acid test sequence or second fungi transcription promoter nucleic acid test sequence may include a detectable moiety. The detectable moiety may be measured to determine the level of transcription initiation or rate of transcription by the test sequence. The detectable moiety may be a protein translated from RNA transcribed from the gene operably linked to the fungi transcription promoter nucleic acid test sequence or to the second fungi transcription promoter nucleic acid test sequence. The detectable moiety may be a RNA transcribed from the gene operably linked to the fungi transcription promoter nucleic acid test sequence or to the second fungi transcription promoter nucleic acid test sequence.

### VI. EXAMPLES

Summary. In these studies disclosed herein, we sought to create the shortest sequences which could fulfill the role of just a core; a sequence which provides a docking site for PIC and can be enhanced by UAS and TFBS. We successfully isolated nineteen strong promoters from a library of candidates comprised of a UAS and a core. These strong promoters were rigorously tested to isolate nine minimal cores shown to be truly modular in nature; they can be combined with both UAS and TFBS isolated from the genome to not only create constitutive promoters, but also, inducible ones. They are highly unique in sequence, bearing no resemblance to any native genomic sequence of S. cerevisiae. They are distinct from each other, spanning a wide range of GC content (47-70%), TFBS, both in quantity and quality present and lastly, they employ different transcriptional activation mechanisms. UAS elements can be identified from libraries and can be combined with core promoter regions to generate short promoters that are as strong or stronger than commonly used native promoters. The synthetic promoters are upwards of ⅙ of the size in DNA.

Experimental Methods.

Strains and Media.

Yeast expression vectors were propagated in Escherichia coli DH10β. E. coli strains were cultivated in LB medium (Sambrook & Russell, 2001) (Teknova) at 37° C. with 225 RPM norbital shaking LB was supplemented with 50 μg/mL ampicillin (Sigma) for plasmid maintenance and propagation. Yeast strains were cultivated on a yeast synthetic complete medium containing 6.7 g of Yeast Nitrogen Base (Difco)/L, 20 g glucose/L and a mixture of amino acids, and nucleotides without uracil (CSM, MP Biomedicals, Solon, Ohio). All medium was supplemented with 1.5% agar for solid media.

For E. coli transformations, 50 μl of electrocompetent E. coli DH10β (Sambrook & Russell, 2001) were mixed with 50 ng of ligated DNA and electroporated (2 mm Electroporation Cuvettes (Bioexpress) with Biorad Genepulser Xcell) at 2.5 kV. Transformants were recovered for one hour at 37° C. in 1 mL SOC Medium (Cellgro), plated on LB agar, and incubated overnight. Single clones were amplified in 2 mL LB medium and incubated overnight at 37° C. Plasmids were isolated (QIAprep Spin Miniprep Kit, Qiagen) and confirmed by sequencing.

For yeast transformations, 20 μL of chemically competent S. cerevisiae BY4741 were transformed with 1 μg of each appropriate purified plasmid according to established protocols, (Hegemann & Heick, 2011) plated on CSM-Ura plates, and incubated for two days at 30° C. Single colonies were picked into 2 mL of CSM-Ura liquid media and incubated at 30° C. Yeast and bacterial strains were stored at −80° C. in 15% glycerol. Plasmids from yeast were isolated using Zymoprep™ Yeast Plasmid Miniprep II kit.

Cloning Procedures.

Restriction enzyme-based plasmid construction schemes are detailed in. Oligonucleotides were purchased from Integrated DNA Technologies (Coralville, Iowa). PCR and double stranding reactions were performed with Phusion DNA Polymerase from New England Biolabs (Ipswich, Mass.) according to manufacturer specifications and the schemes listed in. Digestions were performed according to manufacturer's (NEB) instructions. PCR products and digestions were cleaned with a QIAquick PCR Purification Kit (Qiagen). Phosphatase reactions were performed with Antarctic Phosphatase (NEB) according to manufacturer's instructions and heat-inactivated for 20 min at 65° C. Ligations (T4 DNA Ligase, Fermentas) were performed for 3-18 hrs at 22° C. followed by heat inactivation at 65° C. for 20 min.

Library Preparation.

Libraries were ligated in a 3:1 ligation ratio with 2 μg of backbone in 20 μl reaction volume. Library ligations were desalted for 10 min. on nitrocellulose membrane filters (MF™ 0.025 μm VSWP membrane filters) after 24 hrs of ligation at 16° C. The entire ligation mixture was transformed into freshly prepared electrocompetent E. coli DH10β (Sambrook & Russell, 2001) and plated onto LB plates. E. coli colonies were scraped, and plasmids were isolated (QIAprep Spin Miniprep Kit, Qiagen) and transformed into freshly prepared BY4741. After 48 hrs of flask growth, aliquots of each library covering five times the size of the yeast library in terms of number of cells were stored at −80° C. in 15% glycerol.

Flow Cytometry and FACS.

Yeast colonies were picked in triplicate from glycerol stock, and were grown for 2 days to stationary phase. All yeast cultures were inoculated at an OD of 0.01 and grown to an OD of 0.7-0.9. ΔSpt3 BY4741 (Fischer Scientific) strains under galactose growth were inoculated at OD of 0.1 due to lack of consistent growth at lower OD inoculations. Fluorescence was analyzed (LSRFortessa Flow Cytometer, BD Biosciences. Excitation wavelength: 488 nm, Detection wavelength: 530 nm). An average fluorescence and standard deviation was calculated from the mean values for the biological replicates. Flow cytometry data was analyzed using FlowJo software. Libraries were sorted using BD FACSAria Cell sorter. Sorted cells were grown for 24 hrs at 30° C. in 2 mL CSM-Ura media at 225 rpm. At least ten times the amount of cells were plated onto CSM-Ura as isolated from the sorting. Candidates were picked from these plates.

qPCR Assay.

Yeast cultures were grown to optical density of 0.7 to 0.9 and its RNA was extracted (Quick-RNA Miniprep, Zymo Research Corporation). 2 μg RNA was reverse-transcribed (High Capacity cDNA Reverse Transcription Kit, Applied Biosystems) and quantified in triplicate (SYBR Green PCR Master Mix, Life Technologies) immediately after RNA extraction. Transcript levels were measured relative to that of a housekeeping gene (ALG9) (Viia 7 Real Time PCR Instrument, Life Technologies).

LacZ Assay.

Yeast cultures were grown from triplicate glycerol stock for 2 days. Cultures were inoculated at 0.1 OD and grown overnight to optical density of 0.7 to 0.9. Cells were mixed with appropriate reagents and incubated according to instructions (AB Gal-Screen® System). Chemiluminescent signal was measured with Biotek Cytation 3 imaging reader.

### Example 1

### Spacer Length Determination

In order to create cores which could be successfully combined with UAS and TFBS, we needed to determine the minimal number of nucleotides required in yeast cores between the TATA box and the TSS (transcription start site) to promote successful loading of PIC and thus, transcription initiation by RNAP. In S. cerevisiae, the spacing has been suggested to be 37-90 bp (Russel, 1983, Struhl, 1985). This is peculiar since the structure for yeast RNAP supports a spacing of 30-31 bp (Leuther et al., 1996), the optimal spacing that is found in mammalian promoters (Carninci et al., 2006). Thus, we were curious about the true minimal spacing restrictions, especially since mammals have functioning promoters with spacers as short as 28 bp (Carninci et al., 2006). We created libraries with spacing of 20 (N20), 25 (N25) and 30 (N30) nucleotides using random oligonucleotides. By using a fluorescent reporter, the strengths of the libraries were measured. Interestingly, there is a lengthening in the histogram tails towards higher fluorescence in all libraries when compared to the negative control (no yECitrine). However, N30 library appears to be the only library with a small population shift towards higher fluorescence. Concerned we may be overlooking quality candidates sensitive to an UAS, but non-functioning by themselves, we decided to also create libraries of hybrid candidates of UAS_{CIT} and UAS_{CLB} in an effort to pull functioning candidates from non-functioning ones. We also used expression enhancing terminators known to result in mRNA with a longer half-life in order to draw out functioning candidates (Curran et al., 2013). Both UAS caused higher fluorescence shifts in all libraries, with the most dramatic shift seen in N30 library. Expression enhancing terminators resulted in higher fluorescence shifts for all libraries tested as well. The top ˜0.15% expressing cells of every library was sorted by fluorescence activated cell sorting (FACS) (FIG. 1A). After sequencing some of the candidates in this sorted population, the candidate core sequences from the N20 and N25 were not chosen for further study. It appears we selected for extremely uncommon ligation events: multiple insertions, which would result in longer candidates and allow for more variability in sequence. Interestingly, many of these multiple insertions avoided introducing additional TATA boxes. This makes sense since yeast promoters containing multiple non-overlapping TATA boxes are rare, making up only 2% of the all native promoters (Basehoar et al., 2004).

### Example 2

### Candidate Selection

Although all N30 libraries had low frequency of multiple insertions (when compared to N25 and N30 libraries), candidates were only pulled from sorted UAS_{CIT} N30 library since this library had the lowest frequency of multiple insertions. Promoters driving high expression of yECitrine were stripped of their UAS_{CIT}, and the strength of the core by itself was assessed by measuring fluorescence (FIG. 1A). In an effort to isolate which cores could be activated generically, they were combined with UAS_{CLB} and a Gal4 upstream activating sequence (GBS) (FIG. 1A). Cores that did not activate with UAS_{CLB} were removed from the candidate pool.

Unlike UAS_{CLB}, GBS could not be simply placed upstream of the core. A GBS spaced just 5 bp from the core actually reduced expression. Without wishing to bound by any theory, it is proposes that GBS sterically hinders access of PIC to the TATA box. Thus, we distanced GBS slightly further upstream from the TATA box. At 17 bp (the next cloning site upstream), GBS does not result in lower expression levels. However, the expression levels induced by this hybrid were generally low. At 30 bp distance from the TATA box, GBS is able to induce expression, and when combined with certain cores, the level of induced expression is comparable to that of the full native galactose promoter, but at only 22% of the length of full native galactose promoter.

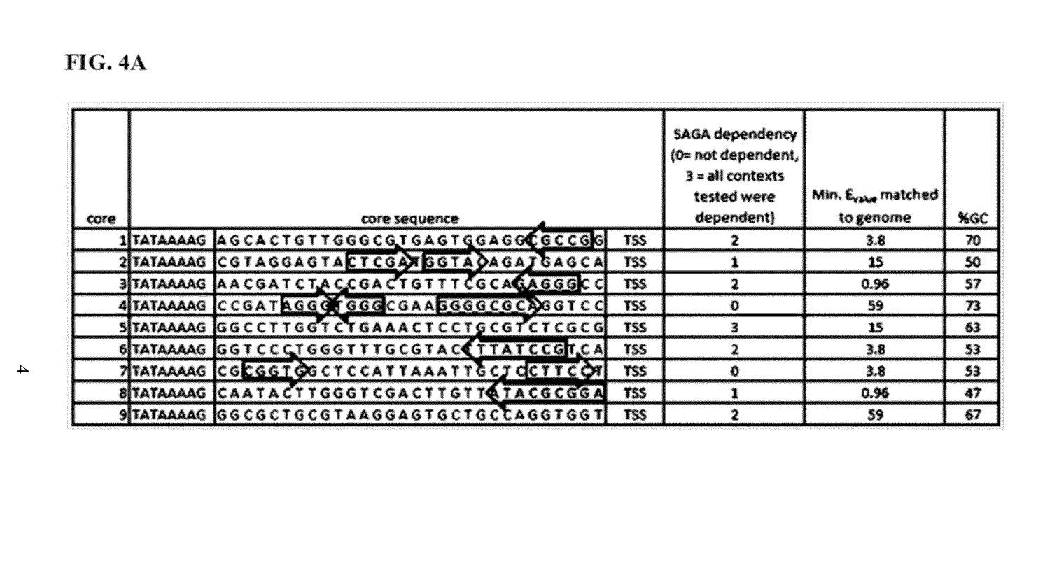

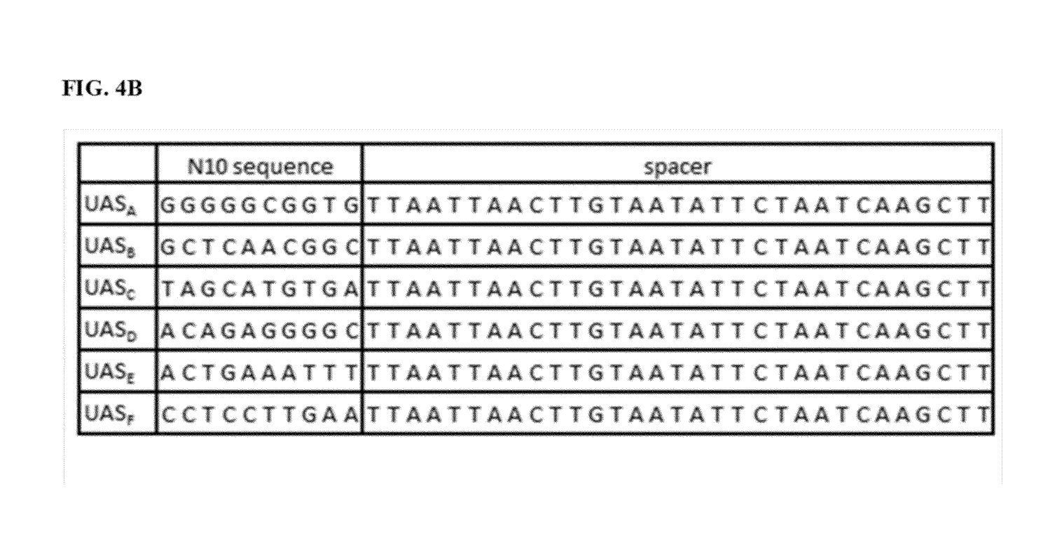

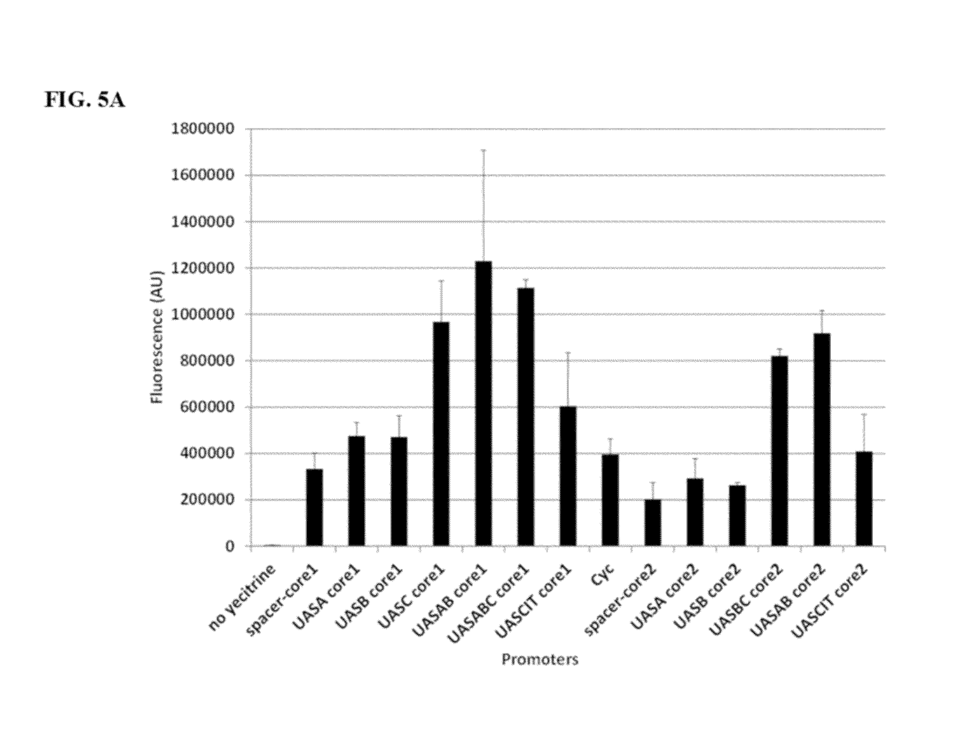

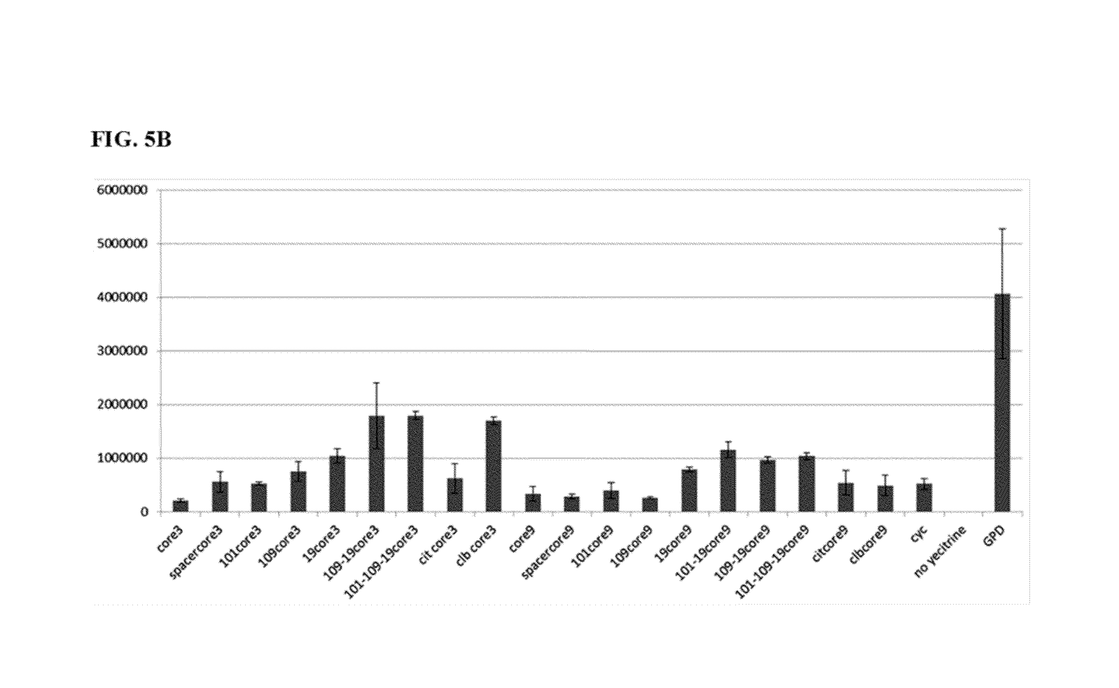

To space GBS 30 bp from the TATA box in the core, an AT-rich spacer was used. This spacer was free of TATA-boxes and TATA-like sequences (any sequence with 2 or less mismatches to TATAW¹AW²R as well as known TFBS (yeastract.com) (FIG. 4B). We show that this spacer has little to no effect on the core's expression levels when grown under glucose. Additionally, the expression driven by the combined spacer and core does not change when the carbon source is altered from glucose to galactose. Thus, any increase in expression is not a result of the spacer itself, but is contributed by the upstream GBS. Above all, if TFBS are to be combined with the cores, sufficient spacing may be required in order to allow loading of PIC and TF.

To determine the context specificity of the cores, they were in situ circumvolved. In situ circumvolution involves removing the expression cassette and introducing it back into the same plasmid location, but in flipped orientation. Thus, sequences originally downstream of the terminator are now upstream of the promoter and vice versa. Compared to Pcyc, the cores were far less affected by this test. When Pcyc was in situ circumvolved, expression was completely abolished. Thus, the cores' behavior can be considered more predictable than that of a commonly used native promoter.

The ability to combine the cores with either a UAS or a TFBS and induce expression highlights the modularity of the cores. This method of hybridization allows for incredible promoter minimization and customization. The cores can be used to create constitutive and inducible promoters.

### Example 3

### Core Analysis and Mechanism of Initiation

The nine selected cores are unique in sequence. They span a wide range of GC content from 47-70% (FIG. 4A). They have a diversity of TFBS, both in quantity and quality based on YEASTRACT database of TFBS (Teixeira et al., 2014) (FIG. 4A). Sequence homology is low among the set, and none of them match to any sequences found in the genome of S. cerevisiae (FIG. 4A). Considering the low level of homology between the nine cores, we were curious about what kinds of initiation mechanisms were being employing. Since all the cores contain a TATA box and generally, TATA-box containing native promoters use the SAGA complex to recruit RNAP, we hypothesized many would use the SAGA complex as well. A critical component of the SAGA complex is its Spt3 subunit. Without it, SAGA-dependent promoters fail to be transcriptionally activated (Bhaumik & Green, 2002, Mohibullah & Hahn, 2008). Thus, to test whether or not promoters created using the cores were recruiting SAGA, we tested their expressions strengths in ΔSpt3 BY4741 strain. Only one core's function was dramatically abolished in all promoters (UAS_{CIT}, UAS_{CLB}, and GBS) (FIG. 4A). The function of two of the cores remained unchanged in all promoter contexts (FIG. 4A). The remaining cores were affected by the knockout of Spt3 differently depending on its promoter contexts (FIG. 4A). While it is difficult to say which cores actually rely on Spt3 due to potential compensatory effects (Stein & Aloy, 2008) and genomic changes (Teng et al., 2013) known to occur in knock out strains, it can be concluded based on the markedly diverse results of removing Spt3 that different transcription initiation machinery is utilized depending on its core and activating partner. The fact that these cores recruit such dramatically different transcription initiation machinery makes them an excellent tool set for promoter engineering efforts.

### Example 4

### Synthetic UAS Isolation and Application

Employing the same spacer used to distance GBS from the core, ten oligonucleotides (N10) were placed 31 bp upstream of core 1 to drive expression of yECitrine. Core 1 was selected because it was shown to be highly activated by GBS. A positive population shift in the histogram was generated by the addition of the ten random nucleotides. 0.01% of the expressing cells were sorted from N10-core3 library using FACS. SEQ ID NO:10, SEQ ID NO:11, SEQ ID NO:12, SEQ ID NO:13, and SEQ ID NO:14 were isolated from this enriched library, and were shown to activate expression of core 1 about three-fold, despite only being comprised of just ten nucleotides. When placed in tandem, the 10 bp isolated UAS offered increased expression of yECitrine. Furthermore, the UAS are generic and can be used to activate other cores. For example, SEQ ID NO:10, SEQ ID NO:11, SEQ ID NO:12, SEQ ID NO:13, and SEQ ID NO:14 were also functional with core 2.

### Example 5

### Synthetic UAS Isolation and Application

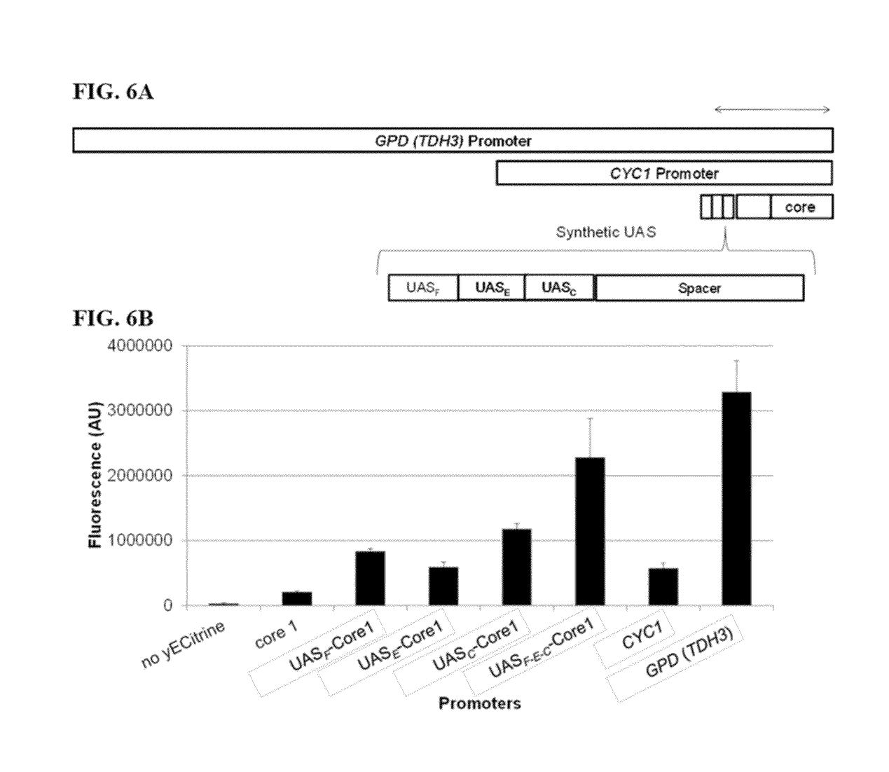

Synthetic hybrid assembled UAS can activate core elements to yield high strength constitutive promoters. As depicted in FIG. 6A, synthetic UAS sequence (e.g., UAS_{F}, UAS_{E} and UAS_{C}) are positioned upstream of core element using AT-rich neutral 30 bp spacer. As depicted in the histogram of FIG. 6B, synthetic UAS sequences can activate core element to strengths of promoters CYC1 and TEF1. Indeed, when hybrid assembled, strengths approaching GPD (TDH3) can be obtained.

### REFERENCES

Alper H, Fischer C, Nevoigt E & Stephanopoulos G (2005) P Natl Acad Sci USA 102: 12678-12683; Bansal M, Kumar A & Yella V R (2014) Current Opinion in Structural Biology 25: 77-85; Basehoar A D, Zanton S J & Pugh B F (2004) Cell 116: 699-709; Bhaumik S R & Green M R (2002) Molecular and Cellular Biology 22: 7365-7371; Blazeck J, Garg R, Reed B & Alper H S (2012) Biotechnology and Bioengineering 109: 2884-2895; Blount B A, Weenink T, Vasylechko S & Ellis T (2012) Plos One 7; Carninci P, Sandelin A, Lenhard B, et al. (2006) Nat Genet 38: 626-635; Curran K A, Karim A S, Gupta A & Alper H S (2013) Metabolic Engineering 19: 88-97; Curran K A, Crook N C, Karim A S, Gupta A, Wagman A M & Alper H S (2014) Nat Commun 5; Du J, Yuan Y, Si T, Lian J & Zhao H (2012) Nucleic Acids Research 40: e142; Hahn S & Young E T (2011) Genetics 189: 705-736; Hammer K, Mijakovic I & Jensen P R (2006). Trends in Biotechnology 24: 53-55; Hegemann J H & Heick S B (2011) Methods in molecular biology (Clifton, N.J.) 765: 189-206; Iyer V & Struhl K (1995) Embo Journal 14: 2570-2579; Jensen P R & Hammer K (1998) Biotechnology and Bioengineering 58: 191-195; Jeppsson M, Johansson B, Jensen P R, Hahn-Hagerdal B & Gorwa-Grauslund M F (2003). Yeast 20: 1263-1272; Khalil Ahmad S, Lu Timothy K, Bashor Caleb J, Ramirez Cherie L, Pyenson Nora C, Joung J K & Collins James J (2012). Cell 150: 647-658; Leuther K K, Bushnell D A & Kornberg R D (1996) Cell 85: 773-779; Liang J, Ning J C & Zhao H (2013) Nucleic Acids Research 41: e54; Ligr M, Siddharthan R, Cross F R & Siggia E D (2006). Genetics 172: 2113-2122; Lubliner S, Keren L & Segal E (2013). Nucleic Acids Research 41: 5569-5581; Mohibullah N & Hahn S (2008). Genes & Development 22: 2994-3006; Nevoigt E, Kohnke J, Fischer C R, Alper H, Stahl U & Stephanopoulos G (2006). Applied and Environmental Microbiology 72: 5266-5273; Raveh-Sadka T, Levo M, Shabi U, Shany B, Keren L, Lotan-Pompan M, Zeevi D, Sharon E, Weinberger A & Segal E (2012). Nat Genet 44: 743-750; Rhee H S & Pugh B F (2012). Nature 483: 295-301; Russel P R (1983). Nature 301: 167-169; Sambrook J & Russell D W (2001). Cold Spring Harbor Laboratory, Cold Spring Harbor, N.Y.; Sharon E, Kalma Y, Sharp A, Raveh-Sadka T, Levo M, Zeevi D, Keren L, Yakhini Z, Weinberger A & Segal E (2012). Nat Biotech 30: 521-530; Stein A & Aloy P (2008). FEBS Letters 582: 1245-1250; Struhl WCaK (1985). The EMBO journal 4: 3273-3280; Teixeira M C, Monteiro P T, Guerreiro J F, et al. (2014). Nucleic Acids Research 42: D161-D166; Teng X, Dayhoff-Brannigan M, Cheng W-C, et al. (2013). Molecular Cell 52: 485-494; Zhang Z & Dietrich F S (2005). Nucleic Acids Research 33: 2838-2851.

### VII. EMBODIMENTS

Embodiments disclosed herein include embodiments P1 to P88 following.

### Embodiment P1

An exogenous fungi transcription promoter nucleic acid sequence comprising: (i) an upstream activating nucleic acid sequence; (ii) a core promoter nucleic acid sequence comprising; (a) a fungi TATA box sequence motif; (b) a fungi transcription start site nucleic acid sequence; and (c) a core promoter linker sequence linking said fungi TATA box sequence motif and said fungi transcription start site nucleic acid sequence; and (iii) an upstream spacer nucleic acid sequence linking said upstream activating nucleic acid sequence to said core promoter nucleic acid sequence.

### Embodiment P2

The exogenous fungi transcription promoter nucleic acid sequence of embodiment 1, wherein said fungi TATA box sequence motif comprises the sequence: TATAW¹AW²R, wherein W¹ and W² are independently A or T, and R is A or G.

### Embodiment P3

The exogenous fungi transcription promoter nucleic acid sequence of embodiment P1 or embodiment P2, wherein said fungi TATA box sequence motif comprises the sequence TATAAAAG.

### Embodiment P4

The exogenous fungi transcription promoter nucleic acid sequence of any one of embodiments P1 to P3, wherein said core promoter linker sequence is 25 to 35 nucleotides in length.

### Embodiment P5

The exogenous fungi transcription promoter nucleic acid sequence of any one of embodiments P1 to P4, wherein said core promoter linker sequence is 30 nucleotides in length.

### Embodiment P6

The exogenous fungi transcription promoter nucleic acid sequence of any one of embodiments P1 to P5, wherein about 45% to about 75% of said core promoter linker sequence is guanine or cytosine.

### Embodiment P7

The exogenous fungi transcription promoter nucleic acid sequence of any one of embodiments P1 to P6, wherein said core promoter linker sequence comprises a transcription factor binding site.

### Embodiment P8

The exogenous fungi transcription promoter nucleic acid sequence of any one of embodiments P1 to P7, wherein said core promoter linker sequence comprises the sequence: AGCACTGTTGGGCGTGAGTGGAGGCGCCGG (SEQ ID NO:1), CGTAGGAGTACTCGATGGTACAGATGAGCA (SEQ ID NO:2), AACGATCTACCGACTGTTTCGCAGAGGGCC (SEQ ID NO:3), CCGATAGGGTGGGCGAAGGGGCGCAGGTCC (SEQ ID NO:4), GGCCTTGGTCTGAAACTCCTGCGTCTCGCG (SEQ ID NO:5), GGTCCCTGGGTTTGCGTACTTTATCCGTCA (SEQ ID NO:6), CGCGGTGGCTCCATTAAATTGCTCCTTCCT (SEQ ID NO:7), CAATACTTGGGTCGACTTGTTATACGCGGA (SEQ ID NO:8), or GGCGCTGCGTAAGGAGTGCTGCCAGGTGGT (SEQ ID NO:9).

### Embodiment P9

The exogenous fungi transcription promoter nucleic acid sequence of any one of embodiments P1 to P8, wherein said upstream activating nucleic acid sequence is a non-native upstream activating nucleic acid sequence.

### Embodiment P10

The exogenous fungi transcription promoter nucleic acid sequence of embodiment P9, wherein said non-native upstream activating nucleic acid sequence is 5 to 50 nucleotides in length.

### Embodiment P11

The exogenous fungi transcription promoter nucleic acid sequence of embodiment P9 or embodiment P10, wherein said non-native upstream activating nucleic acid sequence is 10 nucleotides in length.

### Embodiment P12

The exogenous fungi transcription promoter nucleic acid sequence of any one of embodiments P1 to P11, wherein said upstream activating nucleic acid sequence comprises the sequence: GGGGGCGGTG (SEQ ID NO:10), GCTCAACGGC (SEQ ID NO:11), TAGCATGTGA (SEQ ID NO:12), ACAGAGGGGC (SEQ ID NO:13), ACTGAAATTT (SEQ ID NO:14), or CCTCCTTGAA (SEQ ID NO:15).

### Embodiment P13

The exogenous fungi transcription promoter nucleic acid sequence of any one of embodiments P1 to P11, wherein said upstream activating nucleic acid sequence is a transcription factor binding site.

### Embodiment P14

The exogenous fungi transcription promoter nucleic acid sequence of any one of embodiments P1 to P13, wherein said upstream activating nucleic acid sequence is a GAL4 upstream activating sequence, a CIT upstream activating sequence, or a CLB upstream activating sequence.

### Embodiment P15

The exogenous fungi transcription promoter nucleic acid sequence of any one of embodiments P1 to P13, wherein said upstream activating nucleic acid sequence is a full-length GAL4 upstream activating sequence, a full-length CIT upstream activating sequence, or a full-length CLB upstream activating sequence.

### Embodiment P16

The exogenous fungi transcription promoter nucleic acid sequence of any one of embodiments P1 to P8, wherein said upstream activating nucleic acid sequence is a native upstream activating nucleic acid sequence.

### Embodiment P17

The exogenous fungi transcription promoter nucleic acid sequence of any one of embodiments P1 to P16, wherein said upstream activating nucleic acid sequence is a constitutive-upstream activating nucleic acid sequence.

### Embodiment P18

The exogenous fungi transcription promoter nucleic acid sequence of any one of embodiments P1 to P16, wherein said upstream activating nucleic acid sequence is an inducible-upstream activating nucleic acid sequence.

### Embodiment P19

The exogenous fungi transcription promoter nucleic acid sequence of any one of embodiments P1 to P18, wherein said upstream spacer nucleic acid sequence is 10 to 50 nucleotides in length.

### Embodiment P20

The exogenous fungi transcription promoter nucleic acid sequence of any one of embodiments P1 to P19, wherein said upstream spacer nucleic acid sequence is 15 to 35 nucleotides in length.

### Embodiment P21

The exogenous fungi transcription promoter nucleic acid sequence of any one of embodiments P1 to P20, wherein said upstream spacer nucleic acid sequence is 20 to 40 nucleotides in length.

### Embodiment P22

The exogenous fungi transcription promoter nucleic acid sequence of any one of embodiments P1 to P21, wherein said upstream spacer nucleic acid sequence is 20 to 30 nucleotides in length.

### Embodiment P23

The exogenous fungi transcription promoter nucleic acid sequence of any one of embodiments P1 to P22, wherein said upstream spacer nucleic acid sequence is 30 nucleotides in length.

### Embodiment P24

A fungi cell comprising an exogenous fungi transcription promoter nucleic acid sequence of any one of embodiments P1 to P23.

### Embodiment P25

An expression construct comprising an exogenous fungi transcription promoter nucleic acid sequence of any one of embodiments P1 to P23.

### Embodiment P26

A method of testing a fungi core promoter nucleic acid test sequence, said method comprising determining a level of transcription initiation or a rate of transcription of a core promoter nucleic acid test sequence, wherein said core promoter nucleic acid test sequence comprises a fungi TATA box sequence motif, a fungi transcription start site nucleic acid sequence, and a core promoter linker test sequence.

### Embodiment P27

The method of embodiment P26, wherein said method further comprises determining a level of transcription initiation or a rate of transcription of a second core promoter nucleic acid test sequence, said second core promoter nucleic acid test sequence comprising a fungi TATA box sequence motif, a fungi transcription start site nucleic acid sequence, and a second core promoter linker test sequence, wherein said second core promoter linker test sequence is derived from said core promoter nucleic acid linker test sequence.

### Embodiment P28

The method of embodiment P27, wherein said core promoter nucleic acid test sequence and said second core promoter nucleic acid test sequence comprise the same fungi TATA box sequence motif and the same fungi transcription start site nucleic acid sequence.

### Embodiment P29

The method of embodiment P27, wherein said core promoter nucleic acid test sequence has a level of transcription initiation or a rate of transcription greater than a level of transcription initiation or rate of transcription of a control promoter sequence.

### Embodiment P30

The method of embodiment P29, wherein said control is a native promoter nucleic acid sequence.

### Embodiment P31

The method of embodiment P29 or P30, wherein said control is a native CYC1 promoter nucleic acid sequence.

### Embodiment P32

The method of any one of embodiments P26 to P29, said method further comprising determining the sequence of said core promoter nucleic acid test sequence or said second core promoter nucleic acid test sequence.

### Embodiment P33

The method of any one of embodiment P26 to P32, wherein said core promoter nucleic acid test sequence or said second core promoter nucleic acid test sequence comprises a detectable moiety.

### Embodiment P34

The method of embodiment P33, wherein said detectable moiety is measured to determine said level of transcription initiation or said rate of transcription.

### Embodiment P35

The method of embodiment P26 to P34, wherein said fungi TATA box sequence motif has the sequence TATAAAAG.

### Embodiment P36

The method of embodiment P27 to P35, wherein said core promoter nucleic acid linker test sequence and said second core promoter nucleic acid linker test sequence are independently 10 to 50 nucleotides in length.

### Embodiment P37

The method of embodiment P27 to P36, wherein said core promoter nucleic acid linker test sequence and said second core promoter nucleic acid linker test sequence are independently 15 to 50 nucleotides in length.

### Embodiment P38

The method of embodiment P27 to P37, wherein said core promoter nucleic acid linker test sequence and said second core promoter nucleic acid linker test sequence are independently 15 to 35 nucleotides in length.

### Embodiment P39

The method of embodiment P27 to P38, wherein said core promoter nucleic acid linker test sequence and said second core promoter nucleic acid linker test sequence are independently 15 nucleotides in length.

### Embodiment P40

The method of embodiment P27 to P39, wherein said core promoter nucleic acid linker test sequence and said second core promoter nucleic acid linker test sequence are independently 20 nucleotides in length.

### Embodiment P41

The method of embodiment P27 to P40, wherein said core promoter nucleic acid linker test sequence and said second core promoter nucleic acid linker test sequence are independently 25 nucleotides in length.

### Embodiment P42

The method of embodiment P27 to P41, wherein said core promoter nucleic acid linker test sequence and said second core promoter nucleic acid linker test sequence are independently 30 nucleotides in length.

### Embodiment P43

The method of embodiment P27 to P42, wherein said core promoter nucleic acid linker test sequence and said second core promoter nucleic acid linker test sequence are independently 35 nucleotides in length.

### Embodiment P44

The method of embodiment P27 to P38, wherein said core promoter nucleic acid linker test sequence and said second core promoter nucleic acid linker test sequence are independently 15, 18, 20, 21, 24, 25, 27, or 30 nucleotides in length.

### Embodiment P45

The method of any one of embodiments P26 to P44, wherein said core promoter nucleic acid test sequence further comprises an upstream activating nucleic acid sequence 5′ to said fungi TATA box sequence motif, and an upstream spacer nucleic acid test sequence linking said upstream activating nucleic acid sequence to said fungi TATA box sequence motif.

### Embodiment P46

The method of embodiment P45, wherein said upstream spacer nucleic acid test sequence is 5 to 50 nucleotides in length.

### Embodiment P47

The method of embodiment P45 or P46, wherein said upstream spacer nucleic acid test sequence is 5 to 40 nucleotides in length.

### Embodiment P48

The method of embodiment P45 to P47, wherein said upstream spacer nucleic acid test sequence is 5 to 30 nucleotides in length.

### Embodiment P49

The method of embodiment P45 to P48, wherein said upstream spacer nucleic acid test sequence is 10 to 40 nucleotides in length.

### Embodiment P50

The method of embodiment P45 to P49, wherein said upstream spacer nucleic acid test sequence is 10 to 30 nucleotides in length.

### Embodiment P51

The method of embodiment P45 to P50, wherein said upstream spacer nucleic acid test sequence is 10 to 20 nucleotides in length.

### Embodiment P52

The method of any one of embodiments P45 to P51, wherein said upstream activating nucleic acid sequence is a non-native upstream activating nucleic acid sequence.

### Embodiment P53

The method of embodiment P52, wherein said non-native upstream activating nucleic acid sequence is 5 to 50 nucleotides in length.

### Embodiment P54

The method of embodiment P52 or P53, wherein said non-native upstream activating nucleic acid sequence is 10 nucleotides in length.

### Embodiment P55

The method of embodiment P52 to P54, wherein said upstream activating nucleic acid sequence has the sequence: GGGGGCGGTG (SEQ ID NO:10), GCTCAACGGC (SEQ ID NO:11), TAGCATGTGA (SEQ ID NO:12), ACAGAGGGGC (SEQ ID NO:13), ACTGAAATTT (SEQ ID NO:14), or CCTCCTTGAA (SEQ ID NO:15).

### Embodiment P56

The method of any one of embodiments P45 to P55, wherein said activating nucleic acid sequence is a transcription factor binding site.

### Embodiment P57

The method any one of embodiments P45 to P56, wherein said upstream activating nucleic acid sequence is a GAL4 upstream activating sequence, a CIT upstream activating sequence, or a CLB upstream activating sequence.

### Embodiment P58

The method of embodiment P45, wherein said upstream activating nucleic acid sequence is a full-length GAL4 upstream activating sequence, a full-length CIT upstream activating sequence, or a full-length CLB upstream activating sequence.

### Embodiment P59

The method of any one of embodiments P45 to P51, wherein said upstream activating nucleic acid sequence is a native upstream activating nucleic acid sequence.

### Embodiment P60

The method of any one of embodiments P45 to P59, wherein said upstream activating nucleic acid sequence is a constitutive-upstream activating nucleic acid sequence.

### Embodiment P61

The method of any one of embodiments P45 to P59, wherein said upstream activating nucleic acid sequence is an inducible-upstream activating nucleic acid sequence.

### Embodiment P62

The method of any one of embodiments P45 to P61, wherein said upstream activating nucleic acid sequence is repeated in tandem.

### Embodiment P63

The method of any one of embodiments P45 to P61, wherein said upstream activating nucleic acid sequence comprises a concatenation of two or more upstream activating nucleic acid sequences.

### Embodiment P64

A method of testing an upstream activating nucleic acid sequence, said method comprising: determining a level of transcription initiation or a rate of transcription of a fungi transcription promoter nucleic acid test sequence comprising a non-native upstream activating nucleic acid test sequence, a fungi promoter sequence, and an upstream spacer nucleic acid test sequence linking said non-native upstream activating nucleic acid test sequence and said fungi promoter sequence.

### Embodiment P65

The method of embodiment P64, wherein said method further comprises determining a level of transcription initiation or a rate of transcription of a second fungi transcription promoter nucleic acid test sequence, said second fungi transcription promoter nucleic acid test sequence comprising a non-native upstream activating nucleic acid test sequence, a fungi promoter sequence, and a second upstream spacer nucleic acid test sequence, wherein said second upstream spacer nucleic acid test sequence is derived from said upstream spacer nucleic acid test sequence.

### Embodiment P66

The method of embodiment P65, wherein said fungi transcription promoter nucleic acid test sequence and said second fungi transcription promoter nucleic acid test sequence comprise the same non-native upstream activating nucleic acid test sequence and the same fungi promoter sequence.

### Embodiment P67

The method of embodiment P65, wherein said upstream activating nucleic acid linker test sequence and said second upstream activating nucleic acid linker test sequence are independently 10 to 100 nucleotides in length.

### Embodiment P68

The method of embodiment P66, wherein said fungi promoter sequence is a native-fungi promoter sequence.

### Embodiment P69

The method of embodiment P66, wherein said fungi promoter sequence is a core promoter nucleic acid sequence comprising; (a) a fungi TATA box sequence motif; (b) a fungi transcription start site nucleic acid sequence; and (c) a core promoter linker sequence linking said fungi TATA box sequence motif and said fungi transcription start nucleic acid sequence.

### Embodiment P70

The method of embodiment P69, wherein said TATA box sequence motif comprises the formula: TATAW¹AW²R, wherein W¹ and W² are independently A or T, and R is A or G.

### Embodiment P71

The method of any one of embodiments P64 to P70, wherein said non-native upstream activating nucleic acid test sequence and said second non-native upstream activating nucleic acid test sequence are independently 5 to 50 nucleotides in length.

### Embodiment P72

The method of any one of embodiments P64 to P71, wherein said non-native upstream activating nucleic acid test sequence and said second non-native upstream activating nucleic acid test sequence are independently 10 nucleotides in length.

### Embodiment P73

The method of any one of embodiments P64 to P72, wherein said non-native upstream activating nucleic acid sequence has the sequence: GGGGGCGGTG (SEQ ID NO:10), GCTCAACGGC (SEQ ID NO:11), TAGCATGTGA (SEQ ID NO:12), ACAGAGGGGC (SEQ ID NO:13), ACTGAAATTT (SEQ ID NO:14), or CCTCCTTGAA (SEQ ID NO:15).

### Embodiment P74

The method of any one of embodiments P64 to P72, wherein said non-native upstream activating nucleic acid sequence is a GAL4 upstream activating sequence, a CIT upstream activating sequence, or a CLB upstream activating sequence.

### Embodiment P75

The method of any one of embodiments P64 to P74, wherein said non-native upstream activating nucleic acid sequence is a constitutive-upstream activating nucleic acid sequence.

### Embodiment P76

The method of any one of embodiments P64 to P75, wherein said non-native upstream activating nucleic acid sequence is an inducible-upstream activating nucleic acid sequence.

### Embodiment P77

The method of any one of embodiments P64 to P76, wherein said level of transcription initiation or said rate of transcription is compared to a control.

### Embodiment P78

The method of any one of embodiments P64 to P77, wherein said control is a native promoter.

### Embodiment P79

The method of any one of embodiments P64 to P77, wherein said control is a native CYC1 promoter.

### Embodiment P80

The method of any one of embodiments P64 to P79, wherein said control is a native upstream activating nucleic acid sequence.

### Embodiment P81

The method of any one of embodiments P64 to P80, wherein said non-native upstream activating nucleic acid sequence is repeated in tandem.

### Embodiment P82

A method of expressing a gene in a fungi cell, said method comprising: (i) transforming a fungi cell with an expression construct comprising a gene operably connected to an exogenous fungi transcription promoter nucleic acid sequence of any one of embodiments P1 to P23; (ii) allowing said fungi cell to express said expression construct, wherein said exogenous fungi transcription promoter nucleic acid sequence modulates a level of transcription initiation or a rate of transcription of said gene, thereby expressing said gene in said fungi cell.

### Embodiment P83

The method of embodiment P82, wherein said gene is an endogenous yeast gene.

### Embodiment P84

The method of embodiment P82, wherein said gene is a heterologous gene.

### Embodiment P85

The method of embodiment P82, wherein said exogenous fungi transcription promoter nucleic acid sequence increases said level of transcription initiation or said rate of transcription of said gene when compared to a control.

### Embodiment P86

The method of embodiment P82, wherein said exogenous fungi transcription promoter nucleic acid sequence decreases said level of transcription initiation or said rate of transcription of said gene when compared to a control.

### Embodiment P87

The method of embodiment P85 or P86, wherein said control is a native promoter.

### Embodiment P88

The method of embodiment P85 or P86, wherein said control is a native CYC1 promoter.

1. An exogenous fungi transcription promoter nucleic acid sequence comprising:
(i) an upstream activating nucleic acid sequence;
(ii) a core promoter nucleic acid sequence comprising;
(a) a fungi TATA box sequence motif;
(b) a fungi transcription start site nucleic acid sequence; and
(c) a core promoter linker sequence linking said fungi TATA box sequence motif and said fungi transcription start site nucleic acid sequence; and
(iii) an upstream spacer nucleic acid sequence linking said upstream activating nucleic acid sequence to said core promoter nucleic acid sequence.

2. (canceled)

3. The exogenous fungi transcription promoter nucleic acid sequence of claim 1, wherein said fungi TATA box sequence motif comprises the sequence TATAAAAG.

4. (canceled)

5. The exogenous fungi transcription promoter nucleic acid sequence of claim 1, wherein said core promoter linker sequence is 30 nucleotides in length.

6. (canceled)

7. The exogenous fungi transcription promoter nucleic acid sequence of claim 1, wherein said core promoter linker sequence comprises a transcription factor binding site.

8. The exogenous fungi transcription promoter nucleic acid sequence of claim 1, wherein said core promoter linker sequence comprises the sequence:
(SEQ ID NO: 1)
AGCACTGTTGGGCGTGAGTGGAGGCGCCGG,
(SEQ ID NO: 2)
CGTAGGAGTACTCGATGGTACAGATGAGCA,
(SEQ ID NO: 3)
AACGATCTACCGACTGTTTCGCAGAGGGCC,
(SEQ ID NO: 4)
CCGATAGGGTGGGCGAAGGGGCGCAGGTCC,
(SEQ ID NO: 5)
GGCCTTGGTCTGAAACTCCTGCGTCTCGCG,
(SEQ ID NO: 6)
GGTCCCTGGGTTTGCGTACTTTATCCGTCA,
(SEQ ID NO: 7)
CGCGGTGGCTCCATTAAATTGCTCCTTCCT,
(SEQ ID NO: 8)
CAATACTTGGGTCGACTTGTTATACGCGGA,
or
(SEQ ID NO: 9)
GGCGCTGCGTAAGGAGTGCTGCCAGGTGGT.

9. The exogenous fungi transcription promoter nucleic acid sequence of claim 1, wherein said upstream activating nucleic acid sequence is a non-native upstream activating nucleic acid sequence.

10. (canceled)

11. (canceled)

12. The exogenous fungi transcription promoter nucleic acid sequence of claim 1, wherein said upstream activating nucleic acid sequence comprises the sequence:
(SEQ ID NO: 10)
GGGGGCGGTG,
(SEQ ID NO: 11)
GCTCAACGGC,
(SEQ ID NO: 12)
TAGCATGTGA,
(SEQ ID NO: 13)
ACAGAGGGGC,
(SEQ ID NO: 14)
ACTGAAATTT,
or
(SEQ ID NO: 15)
CCTCCTTGAA.

13. (canceled)

14. The exogenous fungi transcription promoter nucleic acid sequence of claim 1, wherein said upstream activating nucleic acid sequence is a GAL4 upstream activating sequence, a CIT upstream activating sequence, or a CLB upstream activating sequence.

15.-23. (canceled)

24. A fungi cell comprising an exogenous fungi transcription promoter nucleic acid sequence of claim 1.

25. An expression construct comprising an exogenous fungi transcription promoter nucleic acid sequence of claim 1.

26. A method of testing a fungi core promoter nucleic acid test sequence, said method comprising determining a level of transcription initiation or a rate of transcription of a core promoter nucleic acid test sequence, wherein said core promoter nucleic acid test sequence comprises a fungi TATA box sequence motif, a fungi transcription start site nucleic acid sequence, and a core promoter linker test sequence.

27. The method of claim 26, wherein said method further comprises determining a level of transcription initiation or a rate of transcription of a second core promoter nucleic acid test sequence, said second core promoter nucleic acid test sequence comprising a fungi TATA box sequence motif, a fungi transcription start site nucleic acid sequence, and a second core promoter linker test sequence, wherein said second core promoter linker test sequence is derived from said core promoter nucleic acid linker test sequence.

28.-31. (canceled)

32. The method of claim 26, said method further comprising determining the sequence of said core promoter nucleic acid test sequence or said second core promoter nucleic acid test sequence.

33. (canceled)

34. (canceled)

35. The method of claim 26, wherein said fungi TATA box sequence motif has the sequence TATAAAAG.

36.-44. (canceled)

45. The method of claim 26, wherein said core promoter nucleic acid test sequence further comprises an upstream activating nucleic acid sequence 5′ to said fungi TATA box sequence motif, and an upstream spacer nucleic acid test sequence linking said upstream activating nucleic acid sequence to said fungi TATA box sequence motif.

46.-51. (canceled)

52. The method of claim 45, wherein said upstream activating nucleic acid sequence is a non-native upstream activating nucleic acid sequence.

53. (canceled)

54. (canceled)

55. The method of claim 52, wherein said upstream activating nucleic acid sequence has the sequence:
(SEQ ID NO: 10)
GGGGGCGGTG,
(SEQ ID NO: 11)
GCTCAACGGC,
(SEQ ID NO: 12)
TAGCATGTGA,
(SEQ ID NO: 13)
ACAGAGGGGC,
(SEQ ID NO: 14)
ACTGAAATTT,
or
(SEQ ID NO: 15)
CCTCCTTGAA.

56.-63. (canceled)

64. A method of testing an upstream activating nucleic acid sequence, said method comprising: determining a level of transcription initiation or a rate of transcription of a fungi transcription promoter nucleic acid test sequence comprising a non-native upstream activating nucleic acid test sequence, a fungi promoter sequence, and an upstream spacer nucleic acid test sequence linking said non-native upstream activating nucleic acid test sequence and said fungi promoter sequence.

65.-68. (canceled)

69. The method of claim 64, wherein said fungi promoter sequence is a core promoter nucleic acid sequence comprising;
(a) a fungi TATA box sequence motif;
(b) a fungi transcription start site nucleic acid sequence; and
(c) a core promoter linker sequence linking said fungi TATA box sequence motif and said fungi transcription start nucleic acid sequence.

70.-71. (canceled)

72. The method of claim 64, wherein said non-native upstream activating nucleic acid sequence has the sequence:
(SEQ ID NO: 10)
GGGGGCGGTG,
(SEQ ID NO: 11)
GCTCAACGGC,
(SEQ ID NO: 12)
TAGCATGTGA,
(SEQ ID NO: 13)
ACAGAGGGGC,
(SEQ ID NO: 14)
ACTGAAATTT,
or
(SEQ ID NO: 15)
CCTCCTTGAA.

73.-88. (canceled)


SEQUENCE LISTING (49 sequences)

SEQ ID NO: 1 - DNA, 30 bp, Artificial Sequence
  Synthetic polynucleotide
agcactgttgggcgtgagtggaggcgccgg

SEQ ID NO: 2 - DNA, 30 bp, Artificial Sequence
  Synthetic polynucleotide
cgtaggagtactcgatggtacagatgagca

SEQ ID NO: 3 - DNA, 30 bp, Artificial Sequence
  Synthetic polynucleotide
aacgatctaccgactgtttcgcagagggcc

SEQ ID NO: 4 - DNA, 30 bp, Artificial Sequence
  Synthetic polynucleotide
ccgatagggtgggcgaaggggcgcaggtcc

SEQ ID NO: 5 - DNA, 30 bp, Artificial Sequence
  Synthetic polynucleotide
ggccttggtctgaaactcctgcgtctcgcg

SEQ ID NO: 6 - DNA, 30 bp, Artificial Sequence
  Synthetic polynucleotide
ggtccctgggtttgcgtactttatccgtca

SEQ ID NO: 7 - DNA, 30 bp, Artificial Sequence
  Synthetic polynucleotide
cgcggtggctccattaaattgctccttcct

SEQ ID NO: 8 - DNA, 30 bp, Artificial Sequence
  Synthetic polynucleotide
caatacttgggtcgacttgttatacgcgga

SEQ ID NO: 9 - DNA, 30 bp, Artificial Sequence
  Synthetic polynucleotide
ggcgctgcgtaaggagtgctgccaggtggt

SEQ ID NO: 10 - DNA, 10 bp, Artificial Sequence
  Synthetic polynucleotide
gggggcggtg

SEQ ID NO: 11 - DNA, 10 bp, Artificial Sequence
  Synthetic polynucleotide
gctcaacggc

SEQ ID NO: 12 - DNA, 10 bp, Artificial Sequence
  Synthetic polynucleotide
tagcatgtga

SEQ ID NO: 13 - DNA, 10 bp, Artificial Sequence
  Synthetic polynucleotide
acagaggggc

SEQ ID NO: 14 - DNA, 10 bp, Artificial Sequence
  Synthetic polynucleotide
actgaaattt

SEQ ID NO: 15 - DNA, 10 bp, Artificial Sequence
  Synthetic polynucleotide
cctccttgaa

SEQ ID NO: 16 - DNA, 17 bp, Artificial Sequence
  Synthetic polynucleotide
cgggcgacagccctccg

SEQ ID NO: 17 - DNA, 17 bp, Artificial Sequence
  Synthetic polynucleotide
cggaagactctcctccg

SEQ ID NO: 18 - DNA, 275 bp, Artificial Sequence
  Synthetic polynucleotide
tagagattactacatattccaacaagaccttcgcaggaaagtatacctaaactaattaaagaaatctccgaagttcgcatttcattgaacggctcaattaatctttgtaaatatgagcgtttttacgttcacattgcctttttttttatgtatttaccttgcatttttgtgctaaaaggcgtcacgtttttttccgccgcagccgcccggaaatgaaaagtatgacccccgctagaccaaaaatacttttgtgttattggaggatcgcaatccct

SEQ ID NO: 19 - DNA, 240 bp, Artificial Sequence
  Synthetic polynucleotide
agtggaattattagaatgaccactactccttctaatcaaacacgcggaaatagccgccaaaagacagattttattccaaatgcgggtaactatttgtataatatgtttacatattgagcccgtttaggaaagtgcaagttcaaggcactaatcaaaaaaggagatttgtaaatatagcgaccgaatcaggaaaaggtcaacaacgaagttcgcgatatggatgaacttcggtgcctgtcc

SEQ ID NO: 20 - DNA, 38 bp, Artificial Sequence
  Synthetic polynucleotide
tataaaagagcactgttgggcgtgagtggaggcgccgg

SEQ ID NO: 21 - DNA, 38 bp, Artificial Sequence
  Synthetic polynucleotide
tataaaagcgtaggagtactcgatggtacagatgagca

SEQ ID NO: 22 - DNA, 38 bp, Artificial Sequence
  Synthetic polynucleotide
tataaaagaacgatctaccgactgtttcgcagagggcc

SEQ ID NO: 23 - DNA, 38 bp, Artificial Sequence
  Synthetic polynucleotide
tataaaagccgatagggtgggcgaaggggcgcaggtcc

SEQ ID NO: 24 - DNA, 38 bp, Artificial Sequence
  Synthetic polynucleotide
tataaaagggccttggtctgaaactcctgcgtctcgcg

SEQ ID NO: 25 - DNA, 38 bp, Artificial Sequence
  Synthetic polynucleotide
tataaaagggtccctgggtttgcgtactttatccgtca

SEQ ID NO: 26 - DNA, 38 bp, Artificial Sequence
  Synthetic polynucleotide
tataaaagcgcggtggctccattaaattgctccttcct

SEQ ID NO: 27 - DNA, 38 bp, Artificial Sequence
  Synthetic polynucleotide
tataaaagcaatacttgggtcgacttgttatacgcgga

SEQ ID NO: 28 - DNA, 38 bp, Artificial Sequence
  Synthetic polynucleotide
tataaaagggcgctgcgtaaggagtgctgccaggtggt

SEQ ID NO: 29 - DNA, 41 bp, Artificial Sequence
  Synthetic polynucleotide
gggggcggtgttaattaacttgtaatattctaatcaagctt

SEQ ID NO: 30 - DNA, 41 bp, Artificial Sequence
  Synthetic polynucleotide
gctcaacggcttaattaacttgtaatattctaatcaagctt

SEQ ID NO: 31 - DNA, 41 bp, Artificial Sequence
  Synthetic polynucleotide
tagcatgtgattaattaacttgtaatattctaatcaagctt

SEQ ID NO: 32 - DNA, 41 bp, Artificial Sequence
  Synthetic polynucleotide
acagaggggcttaattaacttgtaatattctaatcaagctt

SEQ ID NO: 33 - DNA, 41 bp, Artificial Sequence
  Synthetic polynucleotide
actgaaatttttaattaacttgtaatattctaatcaagctt

SEQ ID NO: 34 - DNA, 41 bp, Artificial Sequence
  Synthetic polynucleotide
cctccttgaattaattaacttgtaatattctaatcaagctt

SEQ ID NO: 35 - DNA, 10 bp, Artificial Sequence
  Synthetic polynucleotide
attgcgatgc

SEQ ID NO: 36 - DNA, 10 bp, Artificial Sequence
  Synthetic polynucleotide
tcctagcgag

SEQ ID NO: 37 - DNA, 10 bp, Artificial Sequence
  Synthetic polynucleotide
tgtgcgtaag

SEQ ID NO: 38 - DNA, 10 bp, Artificial Sequence
  Synthetic polynucleotide
tttttgaatg

SEQ ID NO: 39 - DNA, 10 bp, Artificial Sequence
  Synthetic polynucleotide
ggatagattc

SEQ ID NO: 40 - DNA, 10 bp, Artificial Sequence
  Synthetic polynucleotide
tcctagcgag

SEQ ID NO: 41 - DNA, 10 bp, Artificial Sequence
  Synthetic polynucleotide
gccgcttttt

SEQ ID NO: 42 - DNA, 10 bp, Artificial Sequence
  Synthetic polynucleotide
tgtgcgggtg

SEQ ID NO: 43 - DNA, 10 bp, Artificial Sequence
  Synthetic polynucleotide
gggacctttg

SEQ ID NO: 44 - DNA, 13 bp, Artificial Sequence
  Synthetic polynucleotide
cctgtatggcgcc

SEQ ID NO: 45 - DNA, 10 bp, Artificial Sequence
  Synthetic polynucleotide
acagaggggc

SEQ ID NO: 46 - DNA, 12 bp, Artificial Sequence
  Synthetic polynucleotide
gttcaggaggcc

SEQ ID NO: 47 - DNA, 12 bp, Artificial Sequence
  Synthetic polynucleotide
gttgactcggcc

SEQ ID NO: 48 - DNA, 12 bp, Artificial Sequence
  Synthetic polynucleotide
gaggagggggcc

SEQ ID NO: 49 - DNA, 20 bp, Artificial Sequence
  Synthetic polynucleotide
ctccggaccaccgtcgcccg